(19) United States
(12) Patent Application Publication
(10) Pub. No.: US 2022/0401468 A1
(43) Pub. Date: Dec. 22, 2022

(54) Heparanase Inhibitor as Anti-Viral and Immunomodulatory Therapy

(71) Applicant: Washington University, St. Louis, MO (US)

(72) Inventors: Katherine Weilbaecher, St. Louis, MO (US); Jingyu Xiang, St. Louis, MO (US)

(73) Assignee: Washington University, St. Louis, MO (US)

(21) Appl. No.: 17/841,655

(22) Filed: Jun. 15, 2022

Related U.S. Application Data
(60) Provisional application No. 63/210,802, filed on Jun. 15, 2021.

(51) Int. Cl. A61K 31/727; A61P 31/14; A61P 31/18; A61P 37/06

(52) U.S. Cl. CPC A61K 31/727; A61P 31/14; A61P 31/18; A61P 37/06

(57) ABSTRACT

A method of treating an RNA virus infection in a subject in need that includes administering a therapeutically effective amount of a heparanase inhibitor. The RNA virus includes SARS-CoV-1, SARS-CoV-2, HTLV-1, HIV-1, and any combination thereof. The heparanase inhibitor includes heparin, a heparin mimetic, or any combination thereof. In some aspects, the heparanase inhibitor is Roneparstat. In some aspects, the administration of the therapeutically effective amount of the heparanase inhibitor results in the inhibition of infection by the RNA virus and associated inflammatory cytokine production.

### CROSS-REFERENCE TO RELATED APPLICATIONS

This application claims priority from U.S. Provisional Application Ser. No. 63/210,802 filed on Jun. 15, 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE DISCLOSURE

The present disclosure generally relates to compositions and methods for treating COVID-19.

### BACKGROUND OF THE DISCLOSURE

Coronavirus disease 2019 (COVID-19), caused by severe acute respiratory syndrome coronavirus 2 (SARS-CoV-2), has quickly become a leading cause of death worldwide. In response to the rapid spreading of COVID-19, a global effort has been poured into the development of novel therapies for the prevention and treatment of the disease. 590+ investigational drugs have entered the pre-IND (Investigational New Drug) stage, with 430+ trials reviewed by the FDA and 9 vaccines deployed around the world). Therapeutic approaches for hospitalized patients with COVID-19 disease include remdesivir, steroids, heparin, and convalescent plasma, which have had some success. However, in contrast to the great success in vaccine development, the progress for effective treatment has been slower.

The complex pathogenesis of COVID-19 consists of two major pathological phases: 1) an early infection phase, characterized by the SARS-CoV-2 viral entry and replication and 2) an inflammation phase, characterized by aberrant pro-inflammatory cytokine release that leads to tissue damage, which can evolve into acute respiratory distress syndrome (ARDS) and hypoxemia that may require mechanical ventilation. About 20% of the COVID-19 patients progress to the severe/critical stage, characterized by a marked increase of inflammatory markers including C-reactive protein, ferritin, and interleukin-6 (IL-6). In these patients, a ‘cytokine storm’ or ‘cytokine release syndrome’ results from excessive non-effective host-immune responses by T-cells, and inflammatory monocytes are associated with mortality, a phenomenon often seen in patients receiving chimeric antigen receptor (CAR) T-cell therapy.

Similar to SARS-CoV and MERS-CoV, the spike (S) glycoprotein is required for the entry of SARS-CoV-2 into host cells. Because of the potent immune response it elicits, the S protein has also been widely used as the target antigen in COVID-19 vaccines. S protein-mediated SARS-CoV-2 entry is co-dependent on host angiotensin-converting enzyme 2 (ACE2) and heparan sulfate; treatment with heparin or its non-anticoagulant derivatives can decrease SARS-CoV-2 entry. Heparan sulfate proteoglycans (HSPGs) are ubiquitously presented glycoproteins comprised of linear, negatively charged polysaccharide chains attached to a variety of cell surface or extracellular matrix proteins. One of the important roles of HSPGs is cell adhesion, which is exploited by many viruses including Herpes simplex virus-1 (HSV-1), Dengue virus (DENV), Human papillomavirus (HPV), and Human T-lymphotropic virus 1 (HTLV-1) to attach to host cells via HSPGs.

The enzyme heparanase is the only known mammalian endoglycosidase capable of degrading HSPGs. Heparanase has been implicated in the pathogenesis of multiple cancers and inflammatory diseases such as pancreatitis, and acute renal injury. In HSV-1-mediated inflammatory disease, heparanase promotes viral shedding and release, and triggers pro-inflammatory cytokine release. A recent clinical study reported an increase of plasma heparanase levels in COVID-19 patients, especially in patients with severe disease. The contribution of heparanase to the pathogenesis of COVID-19 remains nevertheless incompletely characterized.

Due to the elevated expression of heparanase in cancers such as multiple myeloma (MM), several heparanase inhibitors have been developed as anticancer therapies. Roneparstat, also known as SST0001, is a chemically modified, 100% N-acetylated, and glycol-split heparin and a potent inhibitor of heparanase enzyme activity (IC50 [half-maximal inhibitory concentration]=3 nM. Glycol splitting of uronic acid residues within heparin disrupts an essential glucuronic acid that lies within the heparin pentasaccharide responsible for binding to antithrombin. The resulting diminished antithrombin binding activity of the glycol-split heparin greatly diminishes its anticoagulant activity, thus allowing the utilization of the drug at high doses in patients. At the preclinical stage, Roneparstat also showed therapeutic benefits in multiple inflammatory disease models such as acute pancreatitis and acute kidney injury. In a phase I clinical trial in patients with advanced MM, Roneparstat was well tolerated and safe at all doses tested, with no dose-limiting toxicities.

### SUMMARY OF THE DISCLOSURE

In various aspects, a method of treating an RNA virus infection in a subject in need by administration of a therapeutically effective amount of a heparanase inhibitor. The RNA virus includes SARS-CoV-1, SARS-CoV-2, HTLV-1, HIV-1, and any combination thereof. In some aspects, the heparanase inhibitor includes heparin, a heparin mimetic, or any combination thereof. In some aspects, the heparanase inhibitor is Roneparstat. In some aspects, the administration of the therapeutically effective amount of the heparanase inhibitor results in the inhibition of infection by the RNA virus and associated inflammatory cytokine production. In some aspects, the RNA virus is SARS-CoV-2. In some aspects, the RNA virus includes an HSPG-dependent viral entry mechanism.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

The figures described below present various aspects of the disclosure.

FIG. 1A is a schematic representation of a VSV-GFP-ΔG virus modified to express SARS-CoV-2 spike protein.

FIG. 1B is a fluorescence image of Vero-E6 cells infected with the rVSV-S-GFP virus in the presence of 0, 0.012, 0.2, 3.125 μg/mL of SST0001.

FIG. 1C is a fluorescence image of Vero-E6 cells infected with the rVSV-S-GFP virus in the presence of 0, 0.012, 0.2, and 3.125 μg/mL of heparin.

FIG. 1D is a logistic inhibition curve of SST0001 treatment during rVSV-S-GFP infection.

FIG. 1E is a logistic inhibition curve of heparin treatment during rVSV-S-GFP infection.

FIG. 1F contains a series of representative images of immunostaining from SARS-CoV-2 (strain: USA-WA1/2020, 400 PFU) infected monolayers of Vero-E6 cells. Antibodies specific to the SARS-CoV-2 spike (S) or nuclear (N) protein were used to identify SARS-CoV-2 infected cells under various SST0001 treatment conditions (0, 0.0015, 0.097, and 6.25 μg/mL).

FIG. 1G contains a series of representative images of immunostaining from SARS-CoV-2 (strain: USA-WA1/2020, 400 PFU) infected monolayers of Vero-E6 cells. Antibodies specific to the SARS-CoV-2 spike (S) or nuclear (N) protein were used to identify SARS-CoV-2 infected cells under various heparin treatment conditions (0, 0.0015, 0.097, and 6.25 μg/mL).

FIG. 1H is a logistic inhibition curve of SST0001 or heparin treatment during SARS-CoV-2 infection.

FIG. 1I is a logistic inhibition curve of heparin treatment during SARS-CoV-2 infection.

FIG. 1J is a graph summarizing the infectivity of Jurkat-LTR-Luc reporter cells co-cultured with irradiated HTLV-1 producing MT-2 cells in the presence or absence of various doses (10, 100, and 200 μg/mL) of SST0001 for 48 hours; infectivity was quantified via luciferase assay.

FIG. 1K is a graph of the infectivity of HIV-1-LTR-Luc reporter cells that were pretreated overnight with SST0001 (10, 100, 200 μg/mL), followed by the HIV-1 (CXCR4 tropic strain) infection for 24 hours; infectivity was quantified via luciferase assay. Error bars in this figure represent ±SEM. *P<0.05; **P<0.01; ***P<0.001 (2-tailed distribution, homoscedastic student's t-test for 2 groups or 1-way ANOVA for multiple comparison).

FIG. 1L is a graph of the infectivity of HIV-1-LTR-Luc reporter cells that were pretreated overnight with SST0001 (10, 100, 200 μg/mL), followed by the HIV-1 (CCR5 tropic strain) infection for 24 hours; infectivity was quantified via luciferase assay. Error bars in this figure represent ±SEM. *P<0.05; **P<0.01; ***P<0.001 (2-tailed distribution, homoscedastic student's t-test for 2 groups or 1-way ANOVA for multiple comparison).

FIG. 1M is a graph of the infectivity of U87/X4 target cells infected with the VSVg-pseudotyped virus VSVg-HIV-1Δenv-luc for 48 h in the presence or absence of SST0001/Roneparstat (10 and 100 mg/mL). Infectivity (percent) was quantified via luciferase assays. Error bars represent the SEM. *, P, 0.05; **, P, 0.01; ***, P, 0.001; NS, not significant (2-tailed distribution, homoscedastic Student's t-test for 2 groups or 1-way ANOVA for multiple comparisons).

FIG. 1N is a graph of the infectivity of U87/R5 target cells infected with the VSVg-pseudotyped virus VSVg-HIV-1Δenv-luc for 48 h in the presence or absence of Roneparstat (10 and 100 mg/mL). Infectivity (percent) was quantified via luciferase assays. Error bars represent the SEM. *, P, 0.05; **, P, 0.01; ***, P, 0.001; NS, not significant (2-tailed distribution, homoscedastic Student's t-test for 2 groups or 1-way ANOVA for multiple comparisons).

FIG. 2A contains a series of scatter plots summarizing the alignment of major BALF cell clusters by UMAP across control (N=4), moderate (N=3), and severe (N=6) COVID-19 samples (top panel), and a series of UMAP presentations showing the HPSE gene expressions across control, moderate and severe COVID-19 samples (bottom panel).

FIG. 2B is a Heatmap of differential HPSE gene expression of different cell types by each COVID-19 sample. Cell markers (cell types are identified by signature genes) for each cell type are defined as: macrophages: CD68; Mast: TPSB2; B cells: MS4A1; plasma cells: IGHG4; natural killer (NK) cells: KLRD1; neutrophils: FCGR3B; plasmacytoid dendritic cells (pDCs): LILRA4; T cells: CD3D; epithelial: TPPP3, KRT18; myeloid dendritic cells (mDCs): CD1C, CLEC9A.

FIG. 2C is a graph summarizing the expression of HPSE by BALF macrophages across control, moderate and severe COVID-19 samples.

FIG. 2D is a graph summarizing the expression of IL-6 by BALF macrophages across control, moderate and severe COVID-19 samples.

FIG. 2E is a graph summarizing the expression of TNF by BALF macrophages across control, moderate and severe COVID-19 samples.

FIG. 2F is a graph summarizing the expression of IL-1B by BALF macrophages across control, moderate and severe COVID-19 samples.

FIG. 2G is a graph summarizing the expression of CCL2 by BALF macrophages across control, moderate and severe COVID-19 samples.

FIG. 2H is a graph summarizing the correlations between the HPSE expression and the expression of IL-6. Error bars in this figure represent ±SEM. *P<0.05; **P<0.01; ***P<0.001 (2-tailed distribution, homoscedastic student's t-test for 2 groups or 1-way ANOVA for multiple comparison).

FIG. 2I is a graph summarizing the correlations between the HPSE expression and the expressions of TNF. Error bars in this figure represent ±SEM. *P<0.05; **P<0.01; ***P<0.001 (2-tailed distribution, homoscedastic student's t-test for 2 groups or 1-way ANOVA for multiple comparison).

FIG. 2J is a graph summarizing the correlations between the HPSE expression and the expressions of IL-1B. Error bars in this figure represent ±SEM. *P<0.05; **P<0.01; ***P<0.001 (2-tailed distribution, homoscedastic student's t-test for 2 groups or 1-way ANOVA for multiple comparison).

FIG. 2K is a graph summarizing the correlations between the HPSE expression and the expressions of CCL2. Error bars in this figure represent ±SEM. *P<0.05; **P<0.01; ***P<0.001 (2-tailed distribution, homoscedastic student's t-test for 2 groups or 1-way ANOVA for multiple comparison).

FIG. 3A is a schematic diagram summarizing an experimental design to assess human primary monocyte-derived macrophages challenged with SARS-CoV-2 spike (51) protein.

FIG. 3B is a bar graph summarizing qPCR detection of transcription of HPSE, IL-6, TNF, CCL2, and IFNG genes in macrophages challenged with 51 protein (0.5 μg/mL) for 12 hours.

FIG. 3C is a graph of qPCR detection of transcription of IL-6 in macrophages challenged with 51 protein (0.5 μg/mL) or various doses (100, 200, 300 ng/mL) of recombinant active heparanase (rHPSE) for 12 hours.

FIG. 3D is a graph of qPCR detection of transcription of IL-6 genes in macrophages co-treated with 51 protein (0.5 μg/mL) and various doses of SST0001 (50 and 200 μg/mL) for 12 hours.

FIG. 3E is a graph of qPCR detection of transcription of TNF genes in macrophages co-treated with 51 protein (0.5 μg/mL) and various doses of SST0001 (50 and 200 μg/mL) for 12 hours.

FIG. 3F is a graph of qPCR detection of transcription of CCL2 genes in macrophages co-treated with 51 protein (0.5 μg/mL) and various doses of SST0001 (50 and 200 μg/mL) for 12 hours.

FIG. 3G is a graph of qPCR detection of transcription of IFNG genes in macrophages co-treated with 51 protein (0.5 μg/mL) and various doses of SST0001 (50 and 200 μg/mL) for 12 hours.

FIG. 3H is a heat map of inflammatory cytokines detected in conditioned medium collected from macrophages co-treated with 51 protein (0.5 μg/mL) and various doses of SST0001 (100 and 200 μg/mL) for 24 hours. Inflammatory cytokines were quantified using a flow cytometry-based multiplex cytokine array. Fold-change of the secreted inflammatory cytokines were normalized to the untreated control and presented in the heatmap.

FIG. 3I Is a graph of absolute concentrations of secreted TNF-α calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3J is a graph of absolute concentrations of secreted IL-6 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3K is a graph of absolute concentrations of secreted IL-10 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3L is a graph of absolute concentrations of secreted IL-113 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3M is a graph of absolute concentrations of secreted IL-33 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3N is a graph of absolute concentrations of secreted IL-23 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3O is a graph of absolute concentrations of secreted IL-12p70 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3P is a graph of absolute concentrations of secreted IL-17A calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3Q is a graph of absolute concentrations of secreted IL-8 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3R is a graph of absolute concentrations of secreted IL-18 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3S is a graph of absolute concentrations of secreted MCP-1 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3T is a graph of absolute concentrations of secreted IFN-γ calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 3U is a graph of absolute concentrations of secreted IFN-α2 calculated based on standard curve fitting. Error bars represent ±SEM. *P<0.05; **P<0.01; ***P<0.001 (2-tailed distribution, homoscedastic student's t-test for 2 groups or 1-way ANOVA for multiple comparison).

FIG. 4A is a graph summarizing transcription levels of IL6 from human primary macrophages pre-treated with NF-KB inhibitor (BAY) for 1 hour, followed by 51 protein (0.5 μg/mL) challenge for 12 hours; transcription levels were assessed by qPCR. Error bars represent ±SEM. *P<0.05; **P<0.01; ***P<0.001 (2-tailed distribution, homoscedastic student's t-test for 2 groups or 1-way ANOVA for multiple comparison).

FIG. 4B is a graph summarizing transcription levels of TNF from human primary macrophages pre-treated with NF-KB inhibitor (BAY) for 1 hour, followed by 51 protein (0.5 μg/mL) challenge for 12 hours; transcription levels were assessed by qPCR. Error bars represent ±SEM. *P<0.05; **P<0.01; ***P<0.001 (2-tailed distribution, homoscedastic student's t-test for 2 groups or 1-way ANOVA for multiple comparison).

FIG. 4C is a graph summarizing transcription levels of IL1B from human primary macrophages pre-treated with NF-KB inhibitor (BAY) for 1 hour, followed by 51 protein (0.5 μg/mL) challenge for 12 hours; transcription levels were assessed by qPCR. Error bars represent ±SEM. *P<0.05; **P<0.01; ***P<0.001 (2-tailed distribution, homoscedastic student's t-test for 2 groups or 1-way ANOVA for multiple comparison).

FIG. 4D is a graph summarizing transcription levels of IFNG from human primary macrophages pre-treated with NF-KB inhibitor (BAY) for 1 hour, followed by 51 protein (0.5 μg/mL) challenge for 12 hours; transcription levels were assessed by qPCR. Error bars represent ±SEM. *P<0.05; **P<0.01; ***P<0.001 (2-tailed distribution, homoscedastic student's t-test for 2 groups or 1-way ANOVA for multiple comparison).

FIG. 4E contains representative images of NF-KB (p65) immunostained human primary macrophages co-treated with 51 protein (0.5 μg/mL) and various doses of SST0001 (50 and 100 μg/mL) for 24 hours. Top: DAPI; Middle: p65 (Alexa-Fluor-488); Bottom: merged.

FIG. 4F is a graph summarizing the quantification of mean fluorescence intensity (MFI) of the nuclear p65 signals from the images of FIG. 4E; 7-10 high-power fields were captured and quantified for each treatment condition. Error bars represent ±SEM. *P<0.05; **P<0.01; ***P<0.001 (2-tailed distribution, homoscedastic student's t-test for 2 groups or 1-way ANOVA for multiple comparison).

FIG. 4G contains Western blots of cytoplasmic and nuclear p65 in human primary macrophages treated with various doses of SST0001 (50 and 100 μg/mL) and 51 protein (0.5 μg/mL) for 30 min. Blots were re-probed with an anti-TBP antibody as a loading control for nuclear fractions and β-actin for the cytoplasmic fractions.

FIG. 4H is a graph summarizing cytoplasmic and nuclear p65 levels as quantified by NF-KB p65/β-actin ratio; error bars represent ±SEM. *P<0.05; **P<0.01; ***P<0.001 (2-tailed distribution, homoscedastic student's t-test for 2 groups or 1-way ANOVA for multiple comparison).

FIG. 41 is a graph summarizing cytoplasmic and nuclear p65 levels as quantified by p65/TBP ratio; error bars represent ±SEM. *P<0.05; **P<0.01; ***P<0.001 (2-tailed distribution, homoscedastic student's t-test for 2 groups or 1-way ANOVA for multiple comparison).

FIG. 5A is a schematic illustration summarizing a preliminary evaluation of the effects of Roneparstat (SST0001) on SAR-CoV-2 viral infections in an animal model.

FIG. 5B is a graph summarizing changes in body mass observed in the mouse model treated as summarized in FIG. 5A.

FIG. 6A is a representative fluorescence image of Vero-E6 cells infected with VSV-eGFP-SARS-CoV-1 in the presence of 0, 0.012, 0.2, and 3.125 mg/mL of Roneparstat.

FIG. 6B is a representative fluorescence image of Vero-E6 cells infected with VSV-eGFP-SARS-CoV-1 in the presence of 0, 0.012, 0.2, and 3.125 mg/mL of heparin.

FIG. 6C is a Logistic inhibition curve of Roneparstat treatment during VSV-eGFP-SARS-CoV-1 infection.

FIG. 6D is a Logistic inhibition curve of heparin treatment during VSV-eGFP-SARS-CoV-1 infection.

FIG. 7A is a schematic illustration summarizing a microarray analysis of HPSE gene expression in 20-week-old C57BL/6 mice infected by intranasal instillation of 10², 10³, 10⁴, or 10⁵ PFU of SARS-CoV-2 virus.

FIG. 7B is a graph summarizing HPSE gene expression of mice treated as described in FIG. 7A obtained two days after initial infection and grouped by SARS virus dosage.

FIG. 7C is a graph summarizing HPSE gene expression of mice treated as described in FIG. 7A with a viral dose of 10² PFU and grouped by days post-infection.

FIG. 7D is a graph summarizing HPSE gene expression of mice treated as described in FIG. 7A with a viral dose of 10³ PFU and grouped by days post-infection.

FIG. 8A is a graph summarizing the cell viability of cells from the Vero-E6 cell line treated with various doses of Roneparstat (0, 25, 50, 100, and 200 mg/mL) for 24 h; cell viability was examined using a CellTiter-Blue viability assay.

FIG. 8B is a graph summarizing the cell viability of cells from the Jurkat cell line treated with various doses of Roneparstat (0, 25, 50, 100, and 200 mg/mL) for 24 h; cell viability was examined using a CellTiter-Blue viability assay.

FIG. 8C is a graph summarizing the cell viability of cells from the THP-1 cell line treated with various doses of Roneparstat (0, 25, 50, 100, and 200 mg/mL) for 24 h; cell viability was examined using a CellTiter-Blue viability assay.

FIG. 9A is a graph of transcription levels of HPSE expressed by control (shLuc) or HPSE knockdown (KD) THP-1-derived macrophages challenged with 51 protein (0.5 mg/mL) for 12 h; transcription levels were examined by qPCR.

FIG. 9B is a graph of transcription levels of IL6 expressed by control (shLuc) or HPSE knockdown (KD) THP-1-derived macrophages challenged with 51 protein (0.5 mg/mL) for 12 h; transcription levels were examined by qPCR.

FIG. 9C is a graph of transcription levels of IL1B expressed by control (shLuc) or HPSE knockdown (KD) THP-1-derived macrophages challenged with 51 protein (0.5 mg/mL) for 12 h; transcription levels were examined by qPCR.

FIG. 9D is a heat map summarizing increases in inflammatory cytokine detected in a conditioned medium collected from various THP-1 macrophages treated with 51 protein (0.5 mg/mL) for 24 h. Inflammatory cytokines were quantified using a flow cytometry-based multiplex cytokine array and fold changes of the secreted inflammatory cytokines were normalized to the values for the untreated control.

FIG. 9E is a graph summarizing the absolute concentration of secreted IL-6 from the heat map of FIG. 9D, calculated based on standard curve fitting. Error bars represent the SEM. *, P, 0.05; **, P, 0.01; ***, P, 0.001 (2-tailed distribution, homoscedastic Student's t-test for 2 groups or 1-way ANOVA for multiple comparisons).

FIG. 9F is a graph summarizing the absolute concentration of secreted TNF-α from the heat map of FIG. 9D, calculated based on standard curve fitting. Error bars represent the SEM. *, P, 0.05; **, P, 0.01; ***, P, 0.001 (2-tailed distribution, homoscedastic Student's t-test for 2 groups or 1-way ANOVA for multiple comparisons).

FIG. 9G is a graph summarizing the absolute concentration of secreted MCP-1 from the heat map of FIG. 9D, calculated based on standard curve fitting. Error bars represent the SEM. *, P, 0.05; **, P, 0.01; ***, P, 0.001 (2-tailed distribution, homoscedastic Student's t-test for 2 groups or 1-way ANOVA for multiple comparisons).

FIG. 9H is a graph summarizing the absolute concentration of secreted IL-1 b from the heat map of FIG. 9D, calculated based on standard curve fitting. Error bars represent the SEM. *, P, 0.05; **, P, 0.01; ***, P, 0.001 (2-tailed distribution, homoscedastic Student's t-test for 2 groups or 1-way ANOVA for multiple comparisons).

FIG. 10 is a schematic diagram illustrating the dual-targeting actions of Roneparstat during the two pathogenic phases of COVID-19 including the early infection phase, characterized by SARS-CoV-2 viral entry, replication, and spread, and the later inflammation phase, characterized by aberrant proinflammatory cytokine release that leads to tissue damage, ARDS, or death.

Those of skill in the art will understand that the drawings, described below, are for illustrative purposes only. The drawings are not intended to limit the scope of the present teachings in any way.

### DETAILED DESCRIPTION OF THE DISCLOSURE

The present disclosure is based, at least in part, on the discovery that the inhibition of heparanase resulted in decreased viral infection in multiple RNA viruses, including SARS-CoV-2, HTLV-1, and HIV-1, as well as reduced inflammatory cytokine release from macrophages, a key player in cytokine release syndrome associated in COVID-19.

As described in the examples below, the role of heparanase in the pathogenesis of COVID-19 was investigated and Roneparstat was evaluated as a therapy. In line with the role of HSPGs as coreceptors for viral entry, treatment with heparin or Roneparstat effectively decreased the infectivities of SARS-CoV-2 as well as the retroviruses HTLV-1 and HIV-1 in vitro. Using HIV-1Δenv pseudotyped with vesicular stomatitis virus G (VSVg) envelope, an HSPG-independent pseudotyped virus, the antiviral activity of Roneparstat was determined to be specific to HSPG-mediated viral entry. Single-cell RNA sequencing (scRNA-seq) analysis of cells isolated from bronchoalveolar lavage fluid (BALF) of COVID-19 patients showed elevated levels of HPSE gene expression compared to healthy controls; HPSE expression was almost exclusively upregulated in CD68+ macrophages and correlated with disease severity and multiple inflammatory cytokine genes, including IL6, IL1B, and TNF. In primary human macrophages, SARS-CoV-2 S protein challenge induced the expression of HPSE and inflammatory cytokines. The knockdown (KD) of HPSE in human macrophages attenuated the induction of multiple inflammatory cytokines by SARS-CoV-2 S1 protein, suggesting that HPSE plays an important role in the induction of these inflammatory cytokines in macrophages. Further, heparanase blockade with Roneparstat attenuated the production of the inflammatory cytokines in a dose-dependent manner via disruption of NF-KB signaling. Roneparstat was identified as a dual-targeting therapy for COVID-19 to decrease viral infection and dampen the pro-inflammatory immune response mediated by macrophages.

Previous structural analysis revealed that heparin, a structural analog of HS, can bind to the receptor-binding domain (RBD) of the SARS-CoV-2 S1 protein, inducing a distinct conformational change. This interaction between heparin and the SARS-CoV-2 spike protein was also confirmed by competitive binding assays that demonstrated that heparin can bind at high-picomolar affinity As described in the examples below, heparin and Roneparstat potently decreased infection by SARS-CoV-2 in Vero-E6 cells. Using the VSV-eGFP-SARS-CoV-2 chimeric virus, similar potency was observed for Roneparstat or heparin in blocking infectivity, indicating that one of the main mechanisms leading to reduced infectivity could be through Roneparstat's competitive disruption of SARS-CoV-2 spike protein binding to cell surface HS.

In various aspects, administration of Roneparstat reduces the infectivity of other RNA viruses, including, but not limited to, VSV-eGFP-SARS-CoV-1 chimeric virus and pathogenic HIV-1 and HTLV-1. Notably, Roneparstat failed to decrease the infectivity of VSVg-HIV-luc-Δenv, an HSPG-independent pseudotyped HIV-1. These data, described in the examples below, suggest that the reliance on HSPGs to enter the target cell may be a common phenomenon among a variety of RNA viruses.

Heparanase regulates the availability of HS on the cell surface or within the extracellular matrix. Upregulation of heparanase has been primarily studied for its role in cancer progression. In recent years, accumulating evidence suggests that HPSE is also involved in viral disease pathogenesis. In HSV-1-associated disease, the upregulation of HPSE after HSV-1 infection was shown to promote virus shedding and the production of proinflammatory cytokines. It has also been demonstrated previously that cells lacking HPSE expression are intrinsically resistant to HSV-1 infection. In a previously-published corneal infection model, HPSE knockout mice also had decreased virus titers and associated inflammation. Without being limited to any particular theory, the role of heparanase in viral pathogenesis is likely attributable to multiple effects of this enzyme. During the egress of a newly-produced virus from the host cell, the virus may be trapped on the host cell's surface by heparan sulfate-bearing cell surface proteoglycans, including syndecan-1. Heparanase stimulated by viral infection degrades cell surface heparan sulfate chains, thereby facilitating the release of the virus bound to those chains. An additional role for heparanase in viral egress lies in its ability to stimulate the shedding of heparan sulfate proteoglycans from the cell surface. It was previously demonstrated that the expression of heparanase by myeloma cells upregulates extracellular signal-regulated kinase (ERK) signaling, leading to the expression of the syndecan-1 sheddase matrix metalloproteinase 9 (MMP-9). Similarly, during HSV-1 infection, heparanase induces the expression of MMP-3 and MMP-7, which leads to the shedding of cell surface syndecan-1 and the virus that is bound to the heparan sulfate chains.

Previous studies have demonstrated that the addition of purified recombinant heparanase resulted in macrophage activation and the upregulation of inflammatory cytokines, including IL-6. Macrophages from heparanase knockout (Hpa-KO) mice have also been previously shown to express lower levels of inflammatory cytokines (e.g., IL-6, TNF-α, and IL-1β), and the overexpression of Hpse was previously shown to exacerbate inflammatory cytokine production in an ulcerative colitis model. Elevated plasma levels of heparanase were reported as detected in hospitalized patients with COVID-19, but the source of heparanase was not defined.

As described in the examples below, single-cell RNA-seq analysis of cells harvested from BALF was used to identify macrophages as the primary source of heparanase. In a recent report, it was shown that human macrophages were infected by live SARS-CoV-2. Although infection of macrophages by SARS-CoV-2 is restrictive without evidence of replication, mature virions showed long persistence (14 days after initial exposure). In addition, persistent SARS-CoV-2 components in macrophages were demonstrated to induce the production of multiple proinflammatory cytokines such as IL-6, IL-113, and TNF-α. In the examples below, a similar result was demonstrated with the SARS-CoV-2 S1 protein, and HPSE knockdown in THP-1-derived macrophages resulted in a significant attenuation of inflammatory cytokines, highlighting the pivotal role of HPSE in the induction of inflammation. The experimental data described in the examples demonstrate the multifaceted roles of heparanase in both infection and inflammation.

Without being limited to any particular theory, it has been observed that a subset of COVID-19 patients progresses to a severe stage, characterized by dysregulated inflammatory cytokine release that causes tissue injury, acute respiratory distress syndrome (ARDS), and death. The hyperinflammatory state in patients with severe COVID-19 resembles cytokine release syndrome (CRS), which has been observed in patients receiving chimeric antigen receptor (CAR) T-cell therapy. The overproduction of IL-6 by monocytes/macrophages has emerged as a key driver of CRS. Tocilizumab, a humanized monoclonal antibody against the IL-6 receptor, has been evaluated in large randomized clinical trials. While some trials (RECOVERY and REMAP-CAP trials) showed a survival benefit, others, including the COVACTA trial, showed a limited or no benefit in patient mortality. Without being limited to any particular theory, these disparate results may be due to multiple factors, such as the timing of the treatment, the patient population, and the use of glucocorticoids. However, these results may also indicate that targeting a single inflammatory cytokine may not be sufficient to suppress the overall inflammatory disease progression in COVID-19. In the examples below, a cytokine screening of a panel of common proinflammatory cytokines revealed elevations of numerous inflammatory cytokines other than IL-6 after the 51 protein challenge that were blocked by Roneparstat, suggesting that targeting heparanase is a viable therapeutic approach for inhibiting inflammatory cytokine release in COVID-19.

Without being limited to any particular theory, the NF-KB signaling pathway is thought to be the main regulator of the production of inflammatory cytokines during the innate immune response. Previous reports indicate that the SARS-CoV-1 spike protein induces IL-6 and TNF-α production by activating NF-KB. IL-6 is thought to be the main stimulator of STAT3, which allows the full activation of the NF-KB signaling pathway. TNF-α is also known to promote the activation of noncanonical NF-KB signaling. In the case of hyperinflammation, these interactions result in a vicious cycle of NF-KB hyperactivation fueling the production and release of more inflammatory cytokines. As described in the examples below, Western blot assays, as well as immunofluorescence staining of NF-KB (p65) in macrophages, revealed increased p65 expression in both the cytoplasmic and nuclear compartments, suggesting that the spike protein is a strong activator of NF-KB signaling. Treatment with Roneparstat decreased both the cytoplasmic and nuclear p65 levels, suggesting that heparanase inhibition prevented the induction of the expression of p65.

Pulmonary activation of coagulation pathways is common in severe COVID-19, evidenced by the elevated levels of D-dimer and fibrin degradation product (FDP). Patients with severe COVID-19 are at an increased risk of venous thromboembolism and microthrombosis. Disseminated intravascular coagulation (DIC) was reported in 71.4% of nonsurvivors versus 0.6% of survivors among patients diagnosed with COVID-19. Anticoagulant therapy such as heparin was recommended for COVID-19 patients with hypercoagulable syndromes. From one retrospective study, treatment with low-molecular-weight heparin (LMWH) resulted in lower serum IL-6 levels in COVID-19 patients. No therapeutic benefit of heparin was observed in COVID-19 patients with critical disease. In COVID-19 patients with moderate disease, therapeutic doses of heparin increased the probability of survival and reduced the use of cardiovascular or respiratory organ support, compared with the usual-care thromboprophylaxis. The therapeutic dose of heparin, however, is associated with a 10 to 15% risk of significant bleeding. Factors increasing the risk of bleeding include older age, anemia, recent trauma or surgery, hypertension, and renal insufficiency. Many of these risk factors are common in patients with severe COVID-19. In contrast, Roneparstat is a chemically modified heparin that caused little to no bleeding complications in a phase I clinical trial at all doses tested. In our infectivity assay, Roneparstat showed activity comparable to that of unfractionated heparin in the SARS-CoV-2 infectivity assay. An open-label, multicenter phase I clinical trial was carried out to evaluate the safety and tolerability of Roneparstat in patients with relapsed/refractory multiple myeloma. Roneparstat was well tolerated in patients, with no dose-limiting toxicities.

COVID-19 consists of two main pathogenic phases, progressing from the initial infection response phase dominated by SARS-CoV-2 entry and replication to the inflammatory response phase dominated by the host immune response. The current standard COVID-19 therapy includes antiviral therapy with remdesivir and anti-inflammatory drugs such as dexamethasone to decrease disease severity but showed less optimal results in reducing mortality. While cytokines produced during the infection may contribute to the worsening of the disease, they could also be important for the antimicrobial response. Therefore, blocking inflammatory cytokine release alone at the early stage may impair the clearance of the virus and lead to delayed recovery from the disease. This might partially explain the poorer outcomes in mild COVID-19 patients receiving dexamethasone in a previously-published clinical.

The dual-targeting effect of Roneparstat on both viral infection and macrophage-mediated inflammatory cytokine release and its safe profile in patients could be advantageous in this setting. Beyond the scope of COVID-19, HPSE may also play a role in other RNA virus-mediated diseases, such as respiratory syncytial virus (RSV).

FIG. 10 is a schematic diagram illustrating the dual-targeting actions of Roneparstat as a treatment for COVID-19. The pathogenesis of COVID-19 consists of two pathogenic phases: (i) the early infection phase, characterized by SARS-CoV-2 viral entry, replication, and spread, and (ii) the later inflammation phase, characterized by aberrant proinflammatory cytokine release that leads to tissue damage, ARDS, or even death. During the initial infection phase, Roneparstat decreases viral infection by competing with HSPG-dependent viral entry. During the inflammation phase, HPSE blockade via Roneparstat attenuates SARS-CoV-2-mediated inflammatory cytokine release from macrophages, through disruption of NF-KB signaling. As demonstrated in the examples below, Roneparstat provides a dual-targeting therapy for COVID-19 to decrease viral infection and dampen the proinflammatory immune response mediated by macrophages.

In various aspects, the disclosure provides a method for treatment of COVID-19 in a patient in need that comprises administering a therapeutically effective amount of a heparinase inhibitor. The heparinase inhibitor may be any suitable compound including, but not limited to, heparin and heparin mimetic heparinase inhibitors. Non-limiting examples of heparin mimetic heparinase inhibitors include roneparstat (SST0001), a chemically modified 100% N-acetylated and glycol split heparin with very low anticoagulant activity. Without being limited to any particular theory, the administration of the heparinase inhibitor functions as a dual-targeting therapy to decrease the viral infection and dampen the pro-inflammatory immune response mediated by macrophages.

As described in the examples below, heparanase inhibition by administration of an exogenous heparanase inhibitor compound resulted in decreased viral infection in multiple RNA viruses, including SARS-CoV-2, HTLV-1, and HIV-1. In addition, heparanase inhibition reduced inflammatory cytokine release from macrophages, a key player in cytokine release syndrome associated with COVID-19.

Heparanase Modulation Agents

As described herein, HPSE expression has been implicated in various diseases, disorders, and conditions. As such, modulation of HPSE expression (e.g., modulation of heparanase) can be used for the treatment of such conditions. A heparanase modulation agent can modulate heparanase response or induce or inhibit heparanase production. Heparanase modulation can comprise modulating the expression of HPSE on cells, modulating the quantity of cells that express HPSE, or modulating the quality of the HPSE-expressing cells.

Heparanase modulation agents can be any composition or method that can modulate HPSE expression on cells including, but not limited to, macrophages. For example, a heparanase modulation agent can be an activator, an inhibitor, an agonist, or an antagonist. As another example, heparanase modulation can be the result of gene editing.

Heparanase Signal Reduction, Elimination, or Inhibition by Small Molecule Inhibitors, shRNA, siRNA, or ASOs

As described herein, a heparanase modulation agent can be used for use in anti-inflammatory therapy. A heparanase modulation agent can be used to reduce/eliminate or enhance/increase NF-KB signals. For example, a heparanase modulation agent can be a small molecule inhibitor of NF-KB signaling. As another example, a heparanase modulation agent can be a short hairpin RNA (shRNA). As another example, a heparanase modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

Heparanase Inhibiting Agent

One aspect of the present disclosure provides for targeting of heparanase, its receptor, or its downstream signaling. The present disclosure provides methods of treating or preventing viral infection in multiple RNA viruses including, but not limited to, SARS-CoV-2, based on the discovery that heparin, a structural analog of heparin sulfate (HS), can bind to the receptor-binding domain (RBD) of the SARS-CoV-2 Spike (51) protein, inducing a distinct conformation change. Further, administration of heparin or heparin mimetic (SST0001) potently decreases infection with primary SARS-CoV-2 or the rVSV-S-GFP pseudotyped virus in Vero-E6 cells. The present disclosure further provides methods of reducing or preventing inflammatory cytokine release from macrophages, a key player in cytokine release syndrome associated with COVID-19.

As described herein, inhibitors of heparanase (e.g., antibodies, fusion proteins, small molecules) can reduce or prevent viral infection in multiple RNA viruses and associated inflammatory cytokine release. A heparanase inhibitor can be any agent that can inhibit heparanase, downregulate heparanase, or knockdown heparanase.

As an example, a heparanase inhibitor can inhibit NF-KB signaling of inflammatory cytokine release associated with infection by the SARS-CoV-2 virus.

For example, the heparanase inhibitor can be an anti-heparanase antibody. The anti-heparanase antibody can be a murine antibody, a humanized murine antibody, or a human antibody.

As another example, the heparanase inhibitor can be an anti-RBD (receptor binding domain) antibody, wherein the anti-RBD antibody prevents binding of the receptor-binding domain (RBD) of the SARS-CoV-2 Spike (51) protein to its receptor.

As another example, the heparanase inhibitor can be heparin or a heparin mimetic, which has been shown to be a potent and specific inhibitor of viral infection and NF-KB signaling.

As another example, the heparanase inhibitor can be a short hairpin RNA (shRNA) or a short interfering RNA (siRNA) targeting heparanase or the receptor-binding domain (RBD) of the SARS-CoV-2 Spike (51).

As another example, the heparanase inhibitor can be an sgRNA targeting heparanase or the receptor-binding domain (RBD) of the SARS-CoV-2 Spike (S1).

Methods for preparing a heparanase inhibitor (e.g., an agent capable of inhibiting heparanase or the receptor-binding domain (RBD) of the SARS-CoV-2 Spike (S1) can comprise the construction of a protein/Ab scaffold containing the natural RBD receptor as a neutralizing agent; developing inhibitors of the RBD receptor “down-stream”; or developing inhibitors of the heparanase production “up-stream”.

Inhibiting heparanase can be performed by genetically modifying heparanase production in a subject or genetically modifying a subject to reduce or prevent expression of the HPSE gene, such as through the use of CRISPR-Cas9 or analogous technologies, wherein, such modification reduces or prevents heparanase production.

Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula:™=81.5° C.+16.6(log 10[Na+])+0.41(fraction G/C content)−0.63(% formamide)−(600/I). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions 1

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| | Ile, Val, Met, Ala, |
| Leu (L) | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| | Ile, Leu, Met, Phe, |
| Val (V) | Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, heparanase signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of heparanase production by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)20NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for the heparanase inhibitor to target cells by the removal of HPSE signals.

For example, the methods as described herein can comprise a method for altering a target polynucleotide sequence in a cell comprising contacting the polynucleotide sequence with a clustered regularly interspaced short palindromic repeats-associated (Cas) protein.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Therapeutic Methods

Also provided is a process of treating, preventing, or reversing a broad spectrum of RNA viruses including, but not limited to, SARS-CoV-1, SARS-CoV-2, HTLV-1, and HIV-1 that includes administration of a therapeutically effective amount of a heparanase inhibitor. In various aspects, the disclosed process may be used to treat any RNA virus that includes an HSPG-dependent viral entry mechanism without limitation.

In one aspect, COVID-19 may be treated in a subject in need by administration of a therapeutically effective amount of a heparanase inhibitor so as to decrease infection by SARS-CoV-2 and to suppress inflammatory cytokine production in response to SARS-CoV-2 infection.

Methods described herein are generally performed on a subject in need thereof. A subject in need of the therapeutic methods described herein can be a subject having, diagnosed with, suspected of having, or at risk for developing COVID-19 or other RNA virus-related illness as described herein. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of the heparanase inhibitor is, for example, an amount that would cause the desired therapeutic effect in a subject while minimizing undesired side effects. In various embodiments, an effective amount of the heparanase inhibitor described herein can substantially inhibit SARS-CoV-2 or other RNA virus infection and associated inflammatory cytokine production, slow the progress of SARS-CoV-2 or other RNA virus infection and associated inflammatory cytokine production or limit the development of SARS-CoV-2 or other RNA virus infection and associated inflammatory cytokine production.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of the heparanase inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit SARS-CoV-2 or other RNA virus infection and associated inflammatory cytokine production.

In some aspects, a therapeutically effective amount of the heparanase inhibitor comprises less than 400 mg/day of Roneparstat in a human subject. In other aspects, the therapeutically effective amount of the heparanase inhibitor comprises less than 300 mg/day, 250 mg/day, 200 mg/day, 150 mg/day, 100 mg/day, 50 mg/day, 25 mg/day, or 10 mg/day of Roneparstat in a human subject.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treatment can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of the heparanase inhibitor can occur as a single event or over a time course of treatment. For example, the heparanase inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for COVID-19 or other RNA virus infections.

The heparanase inhibitor can be administered simultaneously or sequentially with another agent, such as an antibiotic, an anti-inflammatory, or another agent. For example, the heparanase inhibitor can be administered simultaneously with another agent, such as an antibiotic or an anti-inflammatory. Simultaneous administration can occur through the administration of separate compositions, each containing one or more of the heparanase inhibitor, an antibiotic, an anti-inflammatory, or another agent. Simultaneous administration can occur through the administration of one composition containing two or more of the heparanase inhibitor, an antibiotic, an anti-inflammatory, or another agent. The heparanase inhibitor can be administered sequentially with an antibiotic, an anti-inflammatory, or another agent. For example, the heparanase inhibitor can be administered before or after the administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15 Å.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the heparanase inhibitor. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, and sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory

Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

Experiments involving HTLV-1 were performed under biosafety level 2 (BSL2) practices and procedures. Experiments involving HIV-1 and SARS-CoV-2 were performed at BSL3 facilities under BSL3 practices and procedures. The Jurkat-LTR-Luc reporter line, the HTLV-1-transformed lymphocytic cell line MT-2, and the human monocyte cell line THP-1 (ATCC) were maintained in RPMI 1640 supplemented with 10% fetal bovine serum (FBS). The Vero-E6 (ATCC) cell line and the human 293T cell line were maintained in Dulbecco's modified Eagle's medium (DMEM) supplemented with 10% FBS. The Jurkat-LTR-Luc reporter cell line is a human T-cell leukemia cell line engineered to express firefly luciferase under the control of the HTLV-1 long terminal repeat, as described previously. The TZM-Blue HIV-1 reporter cell line is a HeLa cell clone that expresses CD4, CXCR4, CCR5, and the firefly luciferase gene driven by the HIV-1 long terminal repeat. TZM-Blue and Lenti-X 293T cells (TaKaRa Bio) were maintained in DMEM supplemented with 10% FBS.

Human primary peripheral blood mononuclear cells (PBMCs) were acquired from de-identified donors. PBMCs were isolated using Ficoll-Paque Plus density gradient medium, at a density of 1.077 g/mL (catalog number 10771; Sigma), according to the manufacturer's instructions.

HIV-1 NLHX (CXCR4-tropic) or HIV-1 NLYU2 (CCR5-tropic) viral particles were made by transfecting 293T cells with HIV-1 NLHX or HIV-1 NLYU2 plasm ids, as previously described).

The replication-competent vesicular stomatitis virus (VSV)-SARS-CoV-2 chimeric virus expressing enhanced green fluorescent protein (eGFP) (VSV-eGFP-SARS-CoV-2), in which VSV G was replaced with the SARS-CoV-2 S gene, as well as a similar VSV-SARS-CoV-1 chimeric virus (VSV-eGFP-SARS-CoV-1) was provided by another research laboratory. Both VSV-eGFP-SARS-CoV-2 and VSV-eGFP-SARS-CoV-1 were grown in Vero-E6 cells.

Roneparstat (previously named SST0001 or ¹⁰⁰NA,RO-H) was supplied by Leadiant Biosciences (Rome, Italy). A stock concentration of 50 mg/mL was prepared by solubilizing the drug in sterile water and stored at room temperature (up to 30 days). Heparin sodium salt from porcine intestinal mucosa (catalog number H3149; Sigma) was resuspended in PBS at a stock concentration of 25 mg/mL and stored at 4° C. The NF-KB inhibitor BAY 11-7082 (catalog number B5556; Sigma) was solubilized in dimethyl sulfoxide (DMSO) at a stock concentration of 50 mM and stored at −20° C.

Experiments were analyzed using a two-tailed Student's t-test (2 groups), one-way analysis of variance (ANOVA) (>2 groups or repeated measures), or two-way ANOVA (two variables; the P-value refers to the interaction) using Prism 8 (GraphPad Software, Inc.). A nonparametric Spearman correlation coefficient test was adopted to determine statistically significant correlations between the two groups. Results were considered to reach significance at a P-value of ≤−0.05 and are indicated with asterisks in the figures (*, P≤0.05; **, P≤0.01; ***, P≤0.001; ****, P≤0.0001). Data are presented as mean values; error bars represent the standard errors of the means (SEM).

### Example 1: Effect of Heparanase Inhibitor on Virus Infectivity

To evaluate the effect of a heparanase inhibitor on SARS-CoV-2 infectivity, the following experiments were conducted.

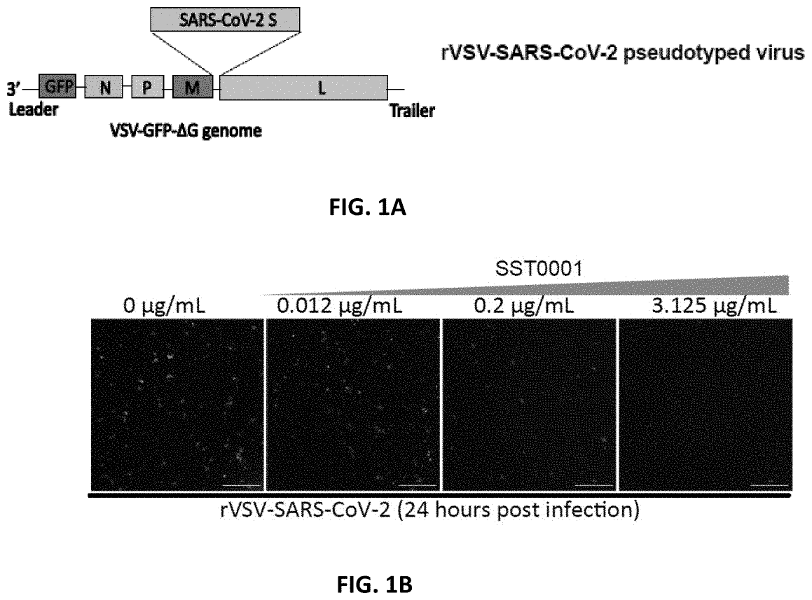

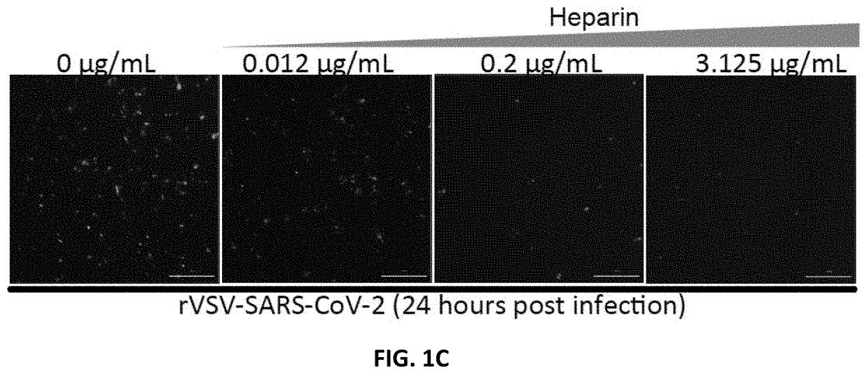

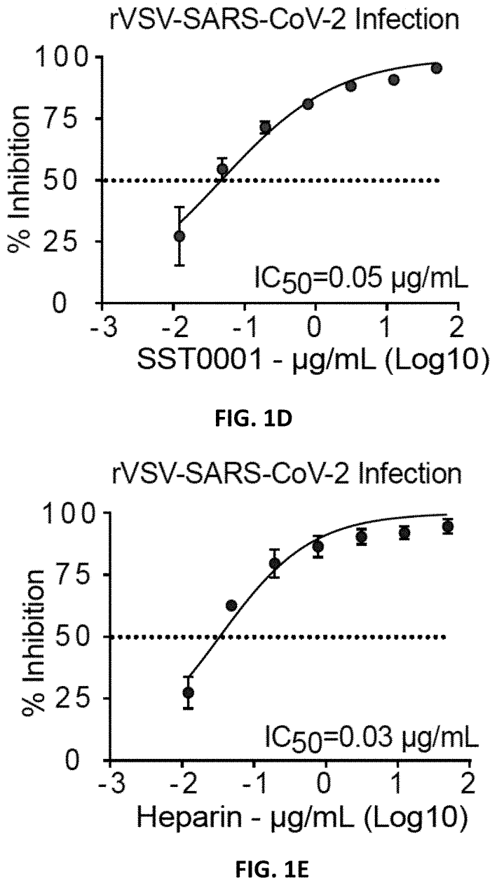

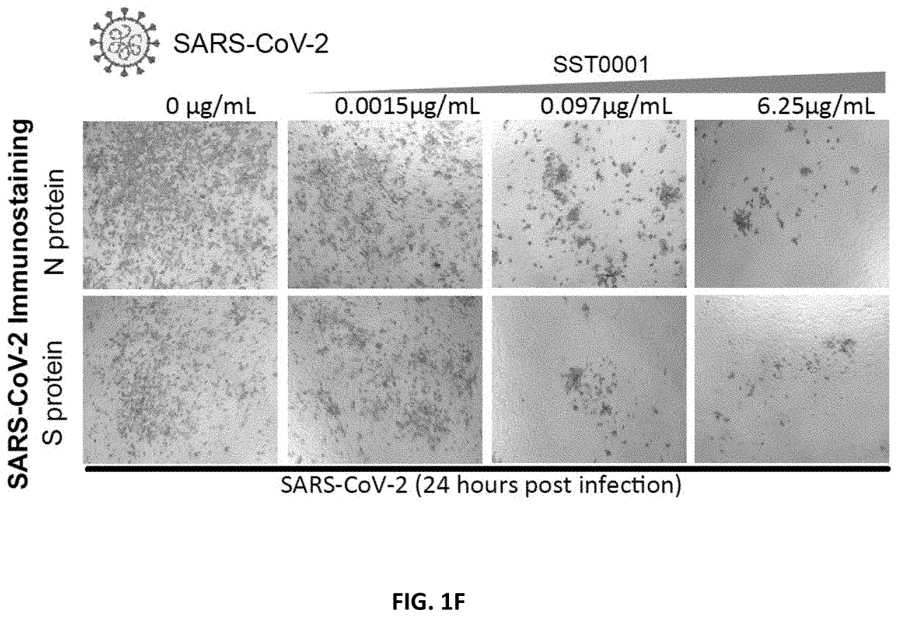

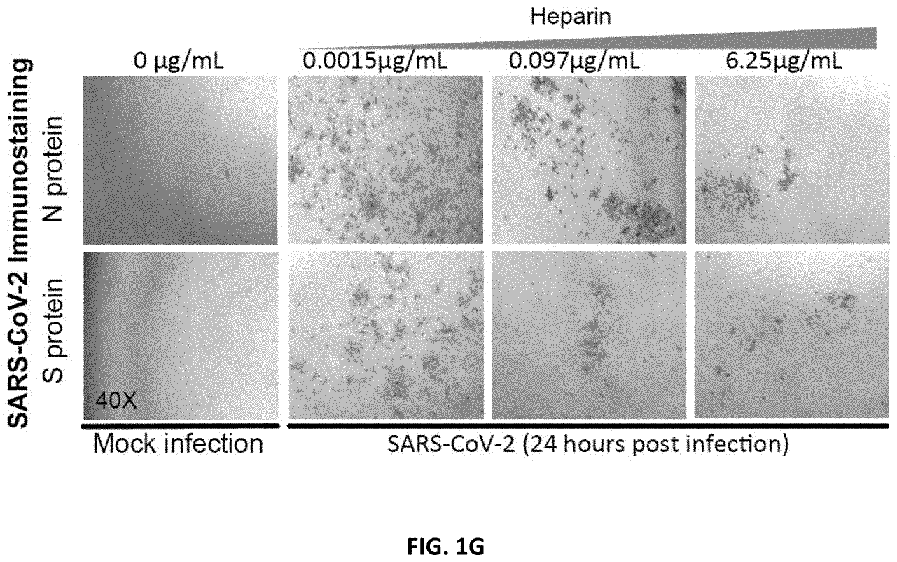

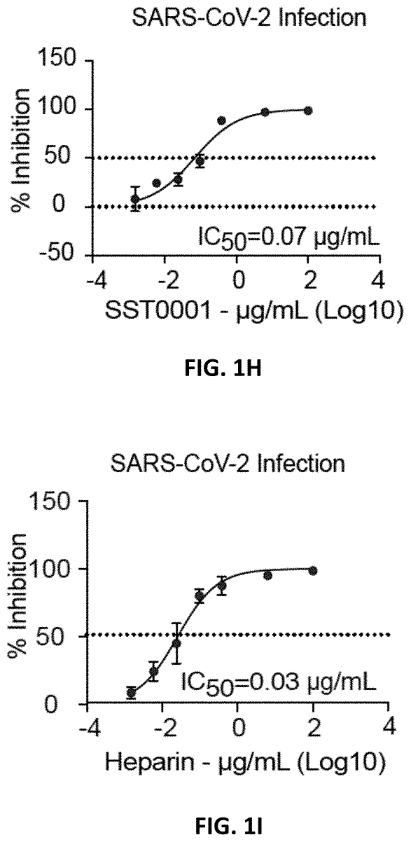

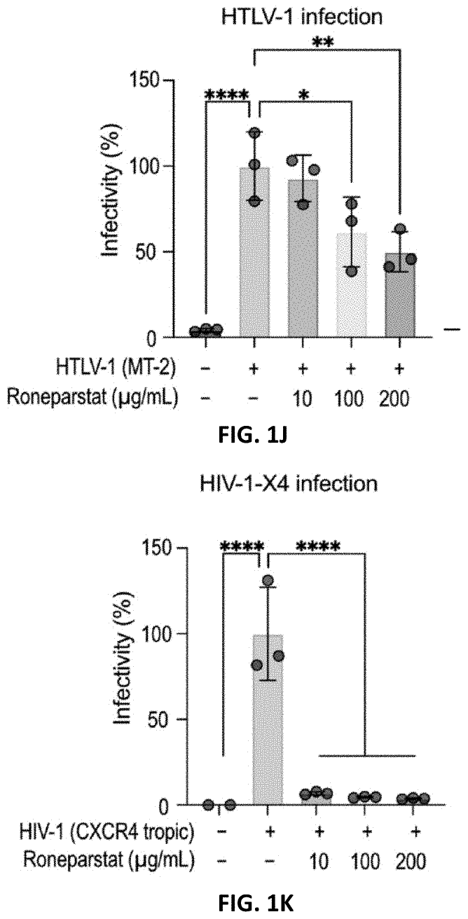

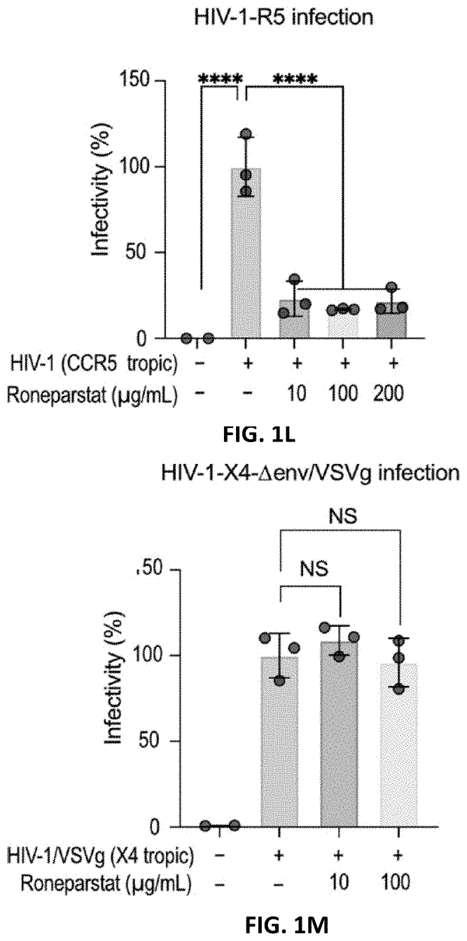

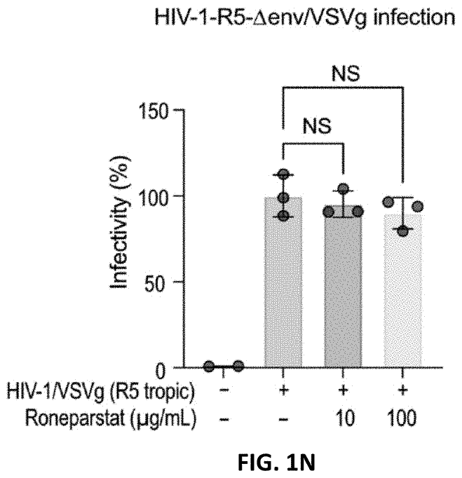

Heparan sulfate (HS) has been identified as an important cofactor for the attachment and entry of multiple viruses, including SARS-CoV-2. A replication-competent VSV-SARS-CoV-2 chimeric virus (VSV-eGFP-SARS-CoV-2), in which the VSV G gene was replaced by the SARS-CoV-2 S gene (see FIG. 1A) was used to infect various cells and the infectivity of the cells was evaluated. VSV G was deleted from the VSV genome, and the SARS-CoV-2 S gene was inserted between the M and L genes in the VSV genome, which resulted in the construction of replication-competent VSV-eGFP-SARS-CoV-2.

SARS-CoV-2 (USA-WA1/2020) was obtained from BEI Resources. USA-WA1/2020 was isolated from an oropharyngeal swab from a COVID-19 patient in Washington. Viral stocks were generated by infecting Vero-E6 cells at an MOI of 0.1 PFU.

In this model, monolayers of Vero-E6 cells were infected by VSV-eGFP-SARS-CoV-2 (multiplicity of infection [MOI]=0.1) in the presence of various doses of Roneparstat or heparin for 1 h. At 24 h post-infection, green fluorescent protein-positive (GFP+) cells, indicative of infection, were quantified using a Cytation 5 microscope.

For the SAR-CoV-2 infectivity assay, both Roneparstat and heparin were subjected to 4-fold serial dilutions from 200 μg/mL to 0.003 μg/m L, and each dilution was mixed with the same volume of SARS-CoV-2 (16,000 PFU/mL). Next, the mixture was transferred to confluent Vero-E6 cells in 48-well plates. The final concentrations of Roneparstat and heparin were ˜100 to 0.0015 μg/m L, and the virus dose was 400 PFU/well. After a 1-h inoculation at 37° C. in a CO2 incubator, the mixture was removed from each well, and fresh DMEM supplemented with 2% FBS and a different dose of Roneparstat or heparin (˜100 to 0.0015 μg/mL) was added (200 μL/well). At 24 h post-infection, the cell culture supernatant from each well was harvested for virus titration by a plaque assay on Vero-E6 cells. The half-maximal inhibitory concentration (IC₅₀) was calculated based on the viral titer from the plaque assay. The infected Vero-E6 cell monolayer was fixed with 300 μL of 4% neutral buffered formaldehyde at room temperature for 2 h, followed by immunostaining with anti-SARS-CoV-2 S or N antibody. Percent inhibition was calculated with the formula % inhibition=100−titer_{treated}/titer_{untreated}×100. The curve fit and IC₅₀ were generated by Prism 8 [log(inhibitor) versus normalized response−variable slope].

For the VSV-SARS-Cov-2 infectivity assay, each compound was diluted serially, 4-fold, from 50 μg/mL to 0.0122 μg/mL and incubated with VSV-eGFP-SARS-CoV-2 or VSV-eGFP-SARS-CoV-1 for 1 h at 37° C. The compound-virus mixtures were used to inoculate Vero-E6 cells (MOI=0.1) in 96-well plates and incubated at 37° C. for 1 h, after which the mixture was replaced with 200 μL of DMEM with 2% FBS containing 50 μg/mL to 0.0122 μg/mL of each compound. At 10 h postinoculation, cells were fixed in 2% formaldehyde for 30 min at room temperature and replaced with PBS. Viral infectivity was measured by automated enumeration of GFP-positive cells from captured images using a Cytation 5 automated fluorescence microscope (BioTek) and analyzed using Gen5 data analysis software (BioTek). Percent inhibition was calculated as 100−green cells with compound treatment/green cells with no compound treatment×100. The IC₅₀ of each peptide was determined by Prism 8 [log(inhibitor) versus normalized response−variable slope].

For the plaque assay, confluent Vero-E6 cells in 12-well plates were infected with 10-fold serial dilutions of SARS-CoV-2, VSV-eGFP-SARS-CoV-2, or VSV-eGFP-SARS-CoV-1 in FBS-free DMEM. After absorption for 1 h at 37° C., the inoculum was removed, and 1 mL of a MEM overlay containing 0.25% low-melting-point agarose, 2% FBS, 0.12% sodium bicarbonate, 25 mM HEPES (pH 7.7), 2 mM L-glutamine, 100 μg/mL of streptomycin, and 100 U/mL of penicillin was added. After incubation at 37° C. for 2 days, wells were fixed with 10% neutral buffered formaldehyde for 2 h, the overlay was removed, the cells were stained with 0.05% (wt/vol) crystal violet, and the plaques were counted.

Treatment with Roneparstat or heparin resulted in a significant dose-dependent decrease in infected cells (FIGS. 1B and 1C), with half-maximal inhibitory concentrations (IC50s) of 0.05 μg/mL and 0.03 μg/m L, respectively (FIGS. 1D and 1E). Similar inhibitory effects on infectivity were observed in Vero-E6 cells infected with the VSV-eGFP-SARS-CoV-1 chimeric virus in which VSV G was replaced with the SARS-CoV-1 S gene (FIGS. 6A, 6B, 6C, and 6D) suggesting that Roneparstat and heparin inhibit cell entry mediated by S protein.

To examine the effect of Roneparstat and heparin during SARS-CoV-2 infection in Vero-E6 cells, Vero-E6 cells were infected with SARS-CoV-2 in the presence of various doses of Roneparstat or heparin for 1 h. At 24 h postinfection, the cell culture supernatant from each well was harvested for virus titration by a plaque assay on Vero-E6 cells. SARS-CoV-2-infected Vero-E6 cells were immunostained with specific anti-spike (S) or -nuclear (N) protein antibody (FIGS. 1F and 1G). In line with the observations from the VSV-eGFP-SARS-CoV-2 infection assay, both Roneparstat and heparin treatment significantly decreased SARS-CoV-2 infection, with 1050s of 0.07 μg/mL and 0.05 μg/m L, respectively (FIGS. 1H and 1I).

Because HSPG is known as one of the coreceptors for the viral entry of SARS-CoV-2 as well as HTLV-1, additional experiments were conducted as described below. The analysis described above was further extended to assess the role of HSPG on the infectivity of retroviruses, including HTLV-1 and HIV-1. Jurkat T cells, engineered to express the luciferase (Luc) gene driven by the HTLV-1 long terminal repeat (LTR), were cocultured with the HTLV-1-producing cell line MT-2 for 48 h in the presence or absence of various doses of Roneparstat. Similarly, TZM-Blue cells engineered to express luciferase driven by the HIV-1 LTR were pretreated with various doses of Roneparstat and infected with HIV-1 for 24 h.

The infectivity of HTLV-1 was measured using the Jurkat-LTR-Luc reporter cell line as described previously. Briefly, the HTLV-1-producing cell line MT-2 was irradiated using an XCe1150 cell irradiator (Kubtec Scientific) at 60 Gy and incubated with Jurkat-LTR-Luc reporters at a ratio of 20,000 MT-2 cells to 100,000 Jurkat-LTR-Luc cells (1:5 ratio). Various doses of Roneparstat were added at the same time of infection. At 24 or 48 h postinfection, cells were collected and lysed in luciferase cell culture lysis buffer (Promega). Luciferase activities were measured and presented as relative light units (RLU).

To assess the infectivity of HIV-1, TZM-Blue reporter cells were pretreated with the inhibitor in a 48-well plate for 24 h, and CXCR4- or CCR5-tropic HIV-1 strains were then added in the presence of various doses of Roneparstat or the vehicle. At 48 h postinfection, infected TZM-Blue cells were collected and lysed with 0.2% Triton X-100 in PBS. Luciferase activities were measured and presented as RLU. For VSVg-HIV-1-luc pseudotyped virus production, 293T cells were cotransfected with pHIV-1-luc/Δenv and pVSVg, and the virus was collected at 72 h posttransfection, as previously described. U87/X4 or U87/R5 cells were seeded into a 48-well plate (2×10⁴ cells per well) and infected with VSVg-HIV-1-luc in the presence or absence of various amounts of Roneparstat. Infected cells were lysed in a solution containing 100 μL PBS and 0.5% Triton X-100 at 48 h postinfection and examined for luciferase activity. Each infection/treatment was run in triplicates.

In these experiments, Roneparstat treatment significantly decreased infection by HTLV-1 (FIG. 1J), as well as both the CXCR4- and CCR5-tropic strains of HIV-1 (FIGS. 1K and 1L). Conversely, infection by VSV was independent of the HSPG-mediated viral entry mechanism.

To confirm that the reduction of infectivity by Roneparstat was HSPG dependent, we constructed HSPG-independent VSVg-HIV-1Δenv pseudotyped virus by cotransfecting HIV-1-luc/Δenv (HIV-1 with defective envelope) with VSVg. CXCR4-expressing U87/X4 or CCR5-expressing U87/R5 target cells were infected with VSVg-HIV-1Δenv-luc in the presence or absence of Roneparstat for 48 h. No reduction of infectivity was observed in either U87/X4 or U87/R5 cells infected with VSVg-HIV-1Δenv-luc (FIGS. 1M and 1N), suggesting that Roneparstat-mediated blockade of infectivity is through HSPGs.

The results of these experiments demonstrated that Roneparstat is potent against SARS-CoV-2 infection in vitro. Further, the results of these experiments demonstrated that treatment with Roneparstat or heparin decreases the infectivity of a broad spectrum of viruses, including SARS-CoV-1, SARS-CoV-2, HTLV-1, and HIV-1, in vitro through an HSPG-dependent viral entry mechanism.

### Example 2: Viability of Cells Treated with Heparanase Inhibitor

To rule out that the reduction of infectivity due to treatment with the heparanase inhibitor described in Example 1 was due to the potential cytotoxicity of Roneparstat, the following experiments were conducted.

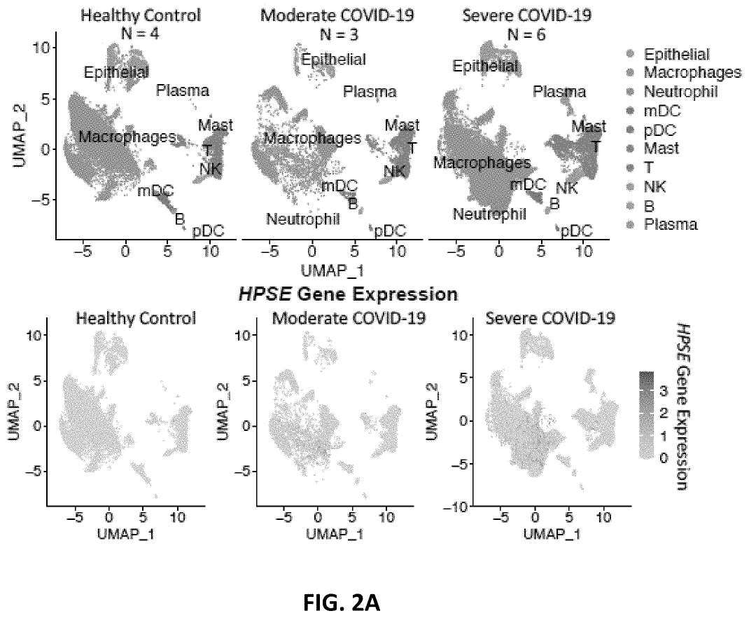

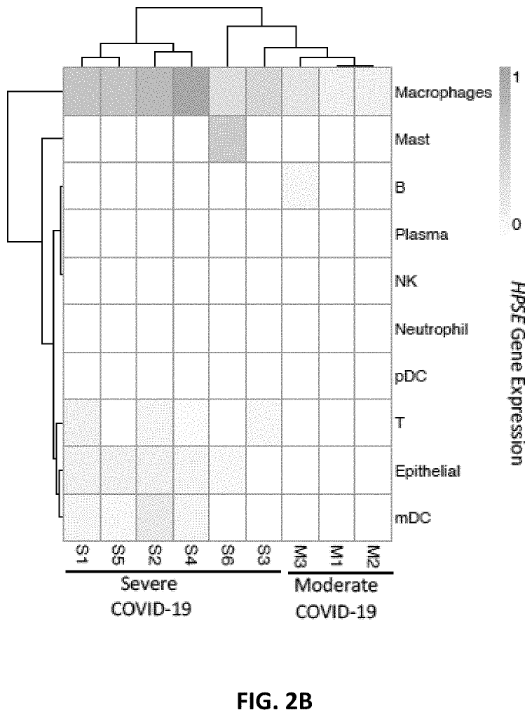

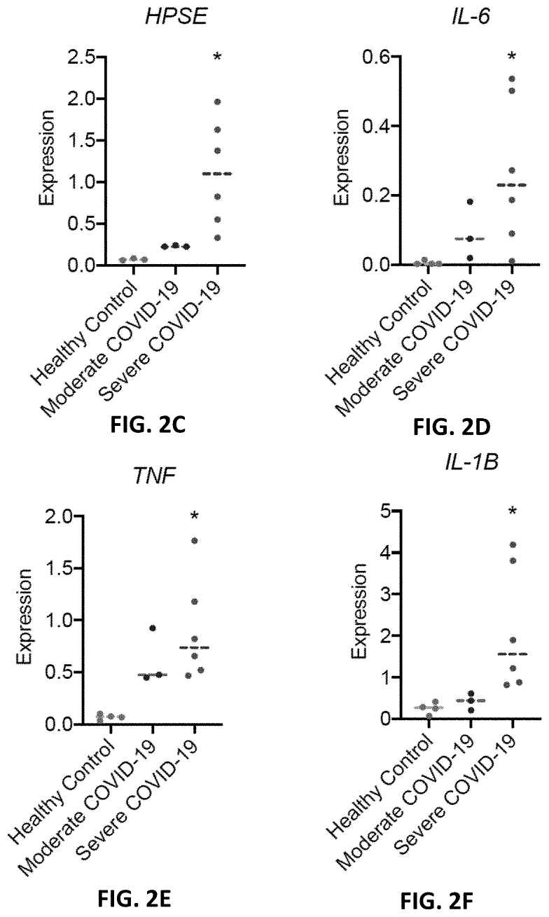

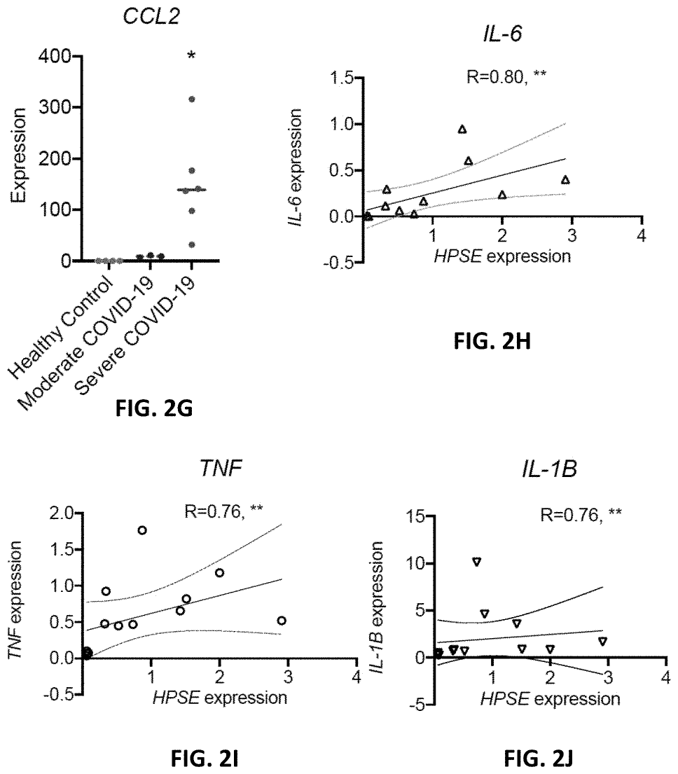

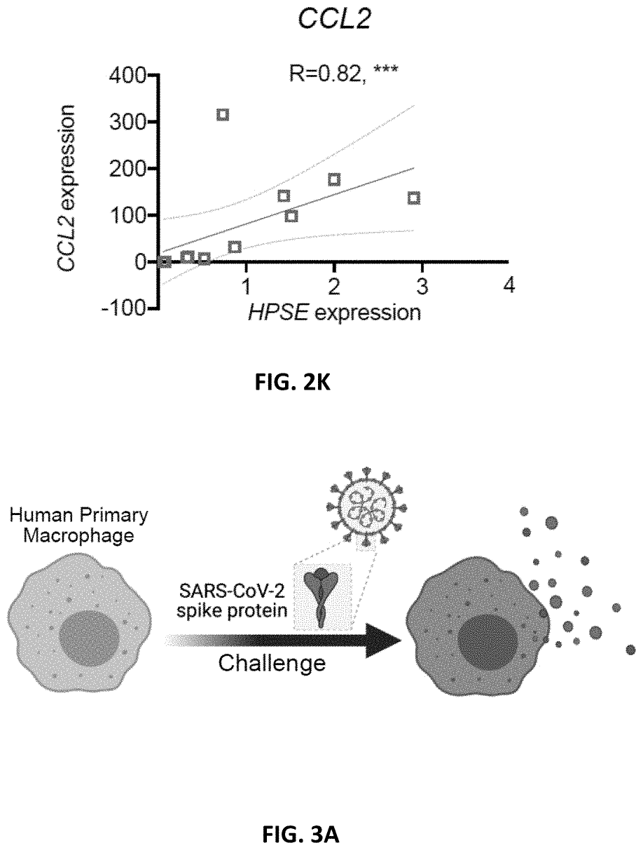

The viability of cells treated with various doses of Roneparstat up to 200 μg/mL was evaluated as described below. The Vero-E6, Jurkat, and THP-1 cell lines were treated with various doses of Roneparstat (0, 25, 50, 100, and 200 μg/mL) for 24 h. Cell viability was examined using a CellTiter-Blue viability assay. As measured by the cell viability assays, no cytotoxicity was observed in multiple cell lines (Vero-E6, Jurkat, and THP-1) treated with Roneparstat (FIGS. 2A, 2B, and 2C).

### Example 3: Upregulation of Heparanase Expression in Pulmonary Macrophages from Covid-19 Patients

Increased plasma levels of heparanase have been reported in COVID-19 patients. To identify the cells that expressed the HPSE gene in COVID-19 patients, the following experiments were conducted.

A publically available microarray dataset (GEO Accession#: GSE33266) of C57BL/6 mice intranasally infected with various doses (10², 10³, 10⁴ or 10⁵ PFU) of SARS-CoV (mouse-adapted strain: MA-15) was analyzed to gain insight on HPSE expression level at the primary site post coronavirus infection (FIG. 7A). The single-cell RNA sequencing (scRNA-seq) data set (GEO accession number GSE145926) from cells harvested from the bronchoalveolar lavage fluid (BALF) of moderate or severe COVID-19 patients and healthy volunteers was analyzed as described below to identify which cells expressed HPSE.

The R package Seurat was used for data scaling, dimensionality reduction, clustering, differential expression analysis, and visualization (58). First, the gene-barcode matrix was normalized using the LogNormalize method in the Seurat NormalizeData function with default parameters. Next, 2,000 variable genes were selected using the vst selection method in the Seurat FindVariableFeatures function. Subsequently, the filtered features were scaled using the Seurat ScaleData function with default parameters, where the two variables nCount_RNA and percent.mito were regressed out during the scaling process. The principal component analysis (PCA) was performed using variable genes, and the top 50 principal components were used to perform UMAP, which maps cells into the two-dimensional space for visualization. Next, a k-nearest-neighbor-based clustering analysis was performed on the PCA-reduced data to identify cell clusters using the Seurat FindClusters function with the resolution set to 1.2. Based on the UMAP reduction results, the DimPlot and FeaturePlot functions in Seurat were respectively used to visualize the BALF cell clusters and highlight the HPSE gene expression levels across healthy control, moderate COVID-19, and severe COVID-19 samples (FIG. 2A).

The FindAllMarkers function in Seurat was used to perform the differential expression analysis. The Model-Based Analysis of Single Cell Transcriptomics (MAST) package was used in FindAllMarkers. For each of the major cell types, the averaged differential HPSE gene expression level was calculated for each COVID-19 sample relative to all of the healthy control cells (FIG. 2B). The HPSE gene in each cell type was considered significant for a COVID-19 sample if the P-value was <0.05, adjusted by the false discovery rate using Bonferroni correction.

Macrophages were reintegrated with a new Seurat object. Macrophages from all samples were integrated using the top 50 dimensions of canonical correlation analysis and PCA, where the parameter k.filter was set to 115. Next, the average expression levels of the HPSE gene and the inflammatory cytokine genes IL6, TNF, IL1B, and CCL2 were calculated using the Seurat AverageExpression function with default parameters. Similarly, average expression levels for each of the healthy control, moderate COVID-19, and severe COVID-19 samples were generated (FIG. 2C).

Microarray analysis of the microarray dataset of SARS-CoV-infected C57BL/6 indicated that the HPSE gene was significantly upregulated in the lung tissue of SARS-CoV (MA-15) infected mice, in a dose-dependent manner (FIGS. 7B, 7C, and 7D).

Single-cell RNA sequencing (scRNA-seq) results from cells harvested from the bronchoalveolar lavage fluid (BALF) of moderate or severe COVID-19 patients and healthy volunteers also indicated that HPSE gene expression was upregulated in various BALF cell populations of patients with COVID-19, especially those with severe COVID-19 (FIG. 2A). Specifically, HPSE was highly expressed in the macrophage population (CD68+), with modest increases in T cells (CD3D+), epithelial cells (TPPP3+KRT18+), and myeloid dendritic cells (mDCs) (CD1C+CLEC9A+) in patients with severe COVID-19 compared to the healthy controls and those with moderate disease (FIG. 2B). Further analysis of the CD68+ macrophage population demonstrated that the expression levels of HPSE, along with inflammatory cytokine genes such as IL6, TNF, IL1B, and CCL2, increased with the disease severity of COVID-19 (FIGS. 2C, 2D, 2E, 2F, and 2G). Inflammatory cytokines such as IL-6 and tumor necrosis factor alpha (TNF-α) are critical players during the inflammation phase of COVID-19. Spearman's correlation coefficient test between HPSE and the inflammatory cytokine genes IL6, TNF, IL1B, and CCL2 showed a significant positive correlation (FIGS. 2H, 2I, 2J, and 2K).

The results of these experiments confirmed that HPSE is upregulated in the macrophages of COVID-19 patients and is associated with inflammatory cytokine release.

### Example 4: Inhibition of HPSE Enzyme Activity with Roneparstat Decreases Inflammatory Cytokine Release Induced by Sars-Cov-2 Spike Protein in Human Primary Macrophages

The binding of SARS-CoV-2 to host cell coreceptors is mediated by the receptor-binding domain (RBD) in the S1 subunit of the spike protein. To test whether S1 protein is sufficient to trigger inflammatory cytokine release and HPSE expression, the following experiments were conducted.

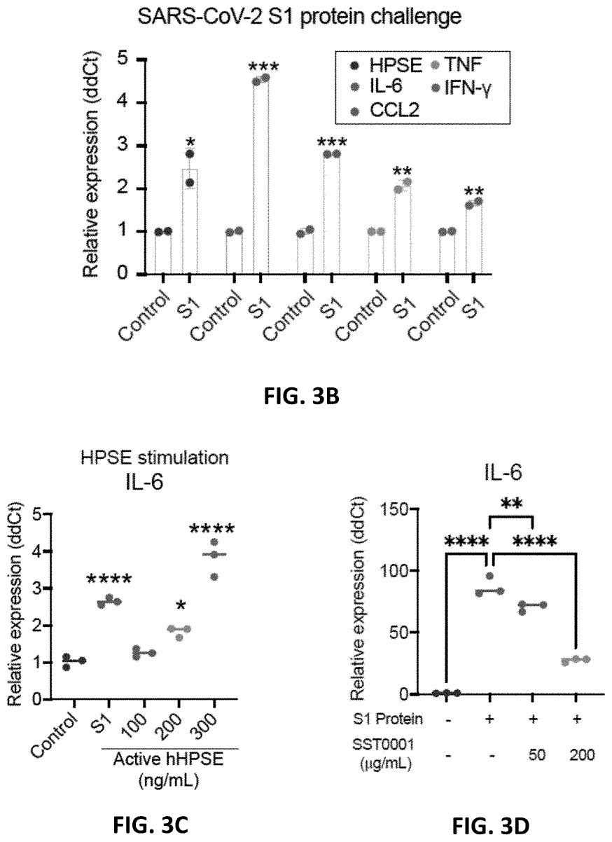

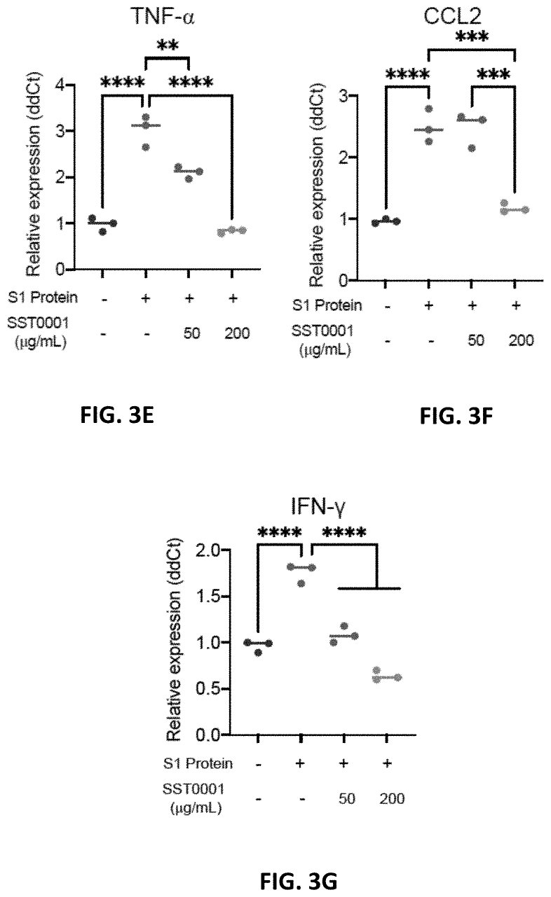

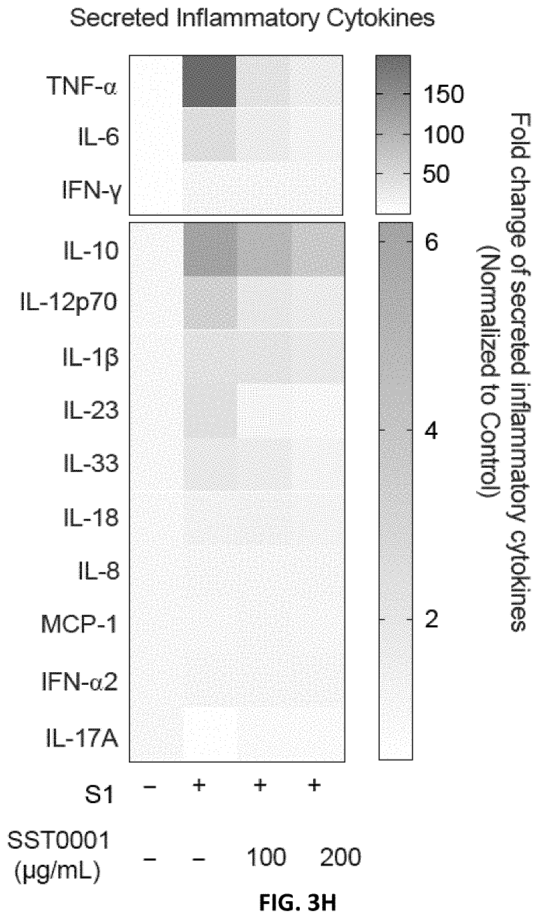

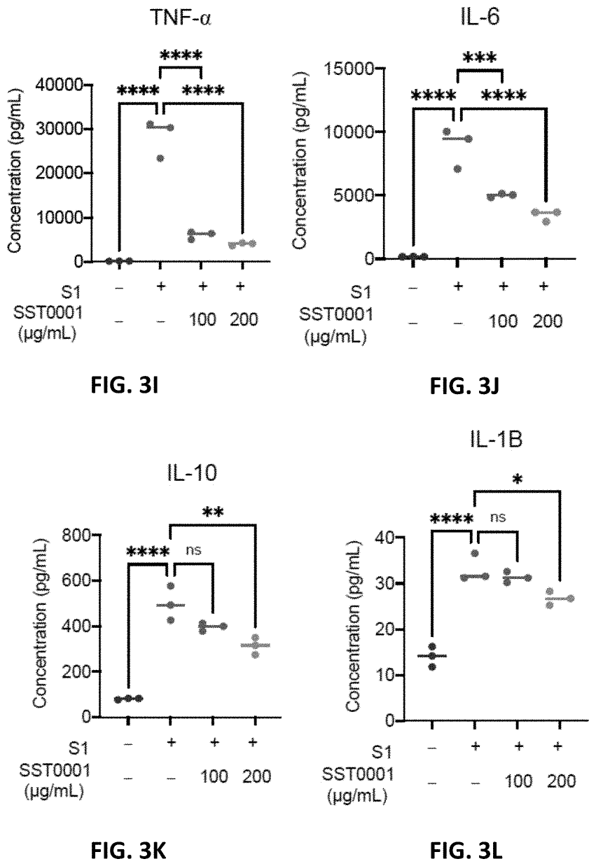

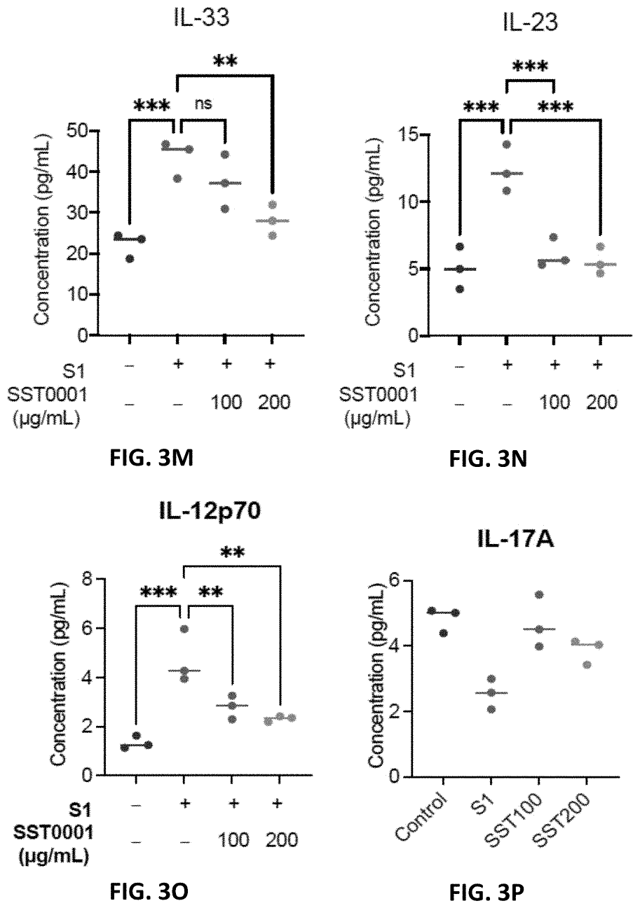

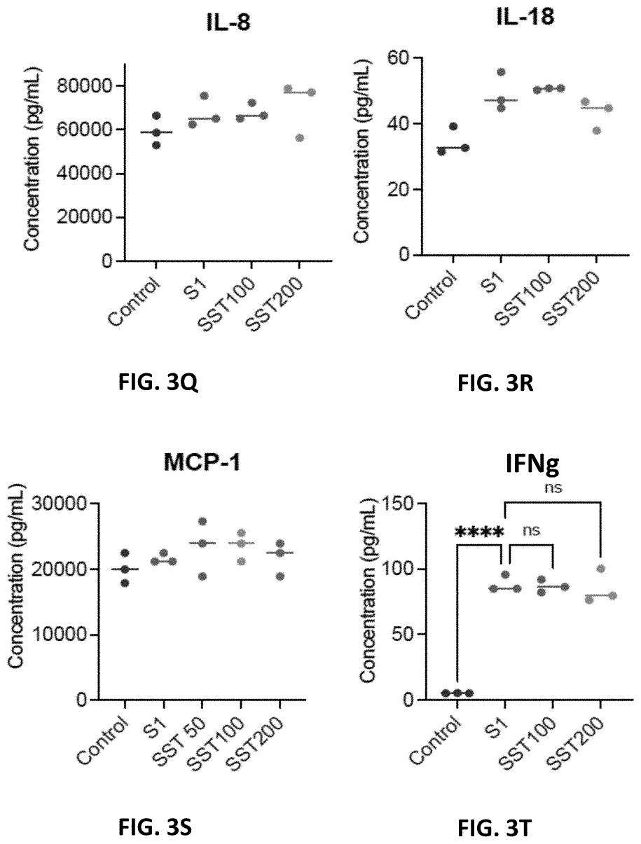

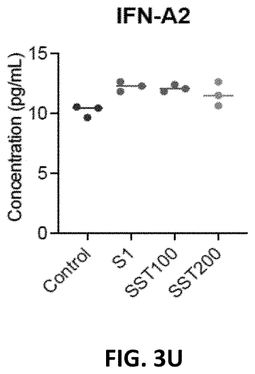

A macrophage challenge model was used in these experiments, in which human primary macrophages were differentiated from monocytes from healthy human donors in vitro and subsequently challenged with SARS-CoV-2 S1 protein overnight (FIG. 3A).

Human CD14⁺ monocytes were purified from human PBMCs of healthy donors using CD14⁺ beads (catalog number 130-050-201; Miltenyi Biotec), according to the manufacturer's instructions. Purified CD14⁺ monocytes were cultured in minimal essential medium alpha (α-MEM) supplemented with 10% FBS and 20 ng/mL human recombinant macrophage colony-stimulating factor (M-CSF) (catalog number 574804; BioLegend). Fresh media with M-CSF were changed every 3 days until the cells reached 70 to 80% confluence (days 12 to 14).

THP-1 macrophage differentiation was initiated by exposing the cells to 5 ng/mL phorbol-12-myristate-13-acetate (PMA) (catalog number 16561-29-8; Sigma-Aldrich) for 72 h. Subsequently, PMA was removed, and THP-1-derived macrophages were washed one time with phosphate-buffered saline (PBS) before the S1 protein (0.5 μg/mL; GenScript, Piscataway, N.J.) challenge. RNA or medium supernatants were harvested 12 h or 24 h after the 51 protein challenge, respectively.

RNA was extracted from cells using an RNeasy kit (Qiagen), and cDNA was generated with qScript cDNA supermix (Quanta Bio). qPCR was performed with PerfeCTa SYBR green supermix reagent (Quanta Bio) on the Bio-Rad CFX96 machine, as previously described. Primers designed for qPCR are as follows: HPSE forward primer 5′-CTCTCTGCTCTGCCATCTTTAG-3′ (SEQ ID NO:1) and reverse primer 5′-CCTCTGGTTGCTATGAGGTTT-3′ (SEQ ID NO:2), IL6 forward primer 5′-CTTCCATCCAGTTGCCTTCT-3′ (SEQ ID NO:3) and reverse primer 5′-CTCCGACTTGTGAAGTGGTATAG-3′ (SEQ ID NO:4), TNF forward primer 5′-CCAGGGACCTCTCTCTAATCA-3′ (SEQ ID NO:5) and reverse primer 5′-TCAGCTTGAGGGTTTGCTAC-3′ (SEQ ID NO:6), CCL2 forward primer 5′-TCATAGCAGCCACCTTCATTC-3′ (SEQ ID NO:7) and reverse primer 5′-CTCTGCACTGAGATCTTCCTATTG-3′ (SEQ ID NO:8), IFNG forward primer 5′-ATGTCCAACGCAAAGCAATAC-3′ (SEQ ID NO:9) and reverse primer 5′-ACCTCGAAACAGCATCTGAC-3′ (SEQ ID NO:10), HPRT forward primer 5′-AGAATGTCTTGATTGTGGAAGA-3′ (SEQ ID NO:11) and reverse primer 5′-ACCTTGACCATCTTTGGATTA-3′ (SEQ ID NO:12), IL1B forward primer 5′-CAAAGGCGGCCAGGATATAA-3′ (SEQ ID NO:13) and reverse primer 5′-CTAGGGATTGAGTCCACATTCAG-3′ (SEQ ID NO:14), and GAPDH forward primer 5′-AGGTCGGTGTGAACGGATTTG-3′ (SEQ ID NO:15) and reverse primer 5′-TGTAGACCATGTAGTTGAGGTCA-3′ (SEQ ID NO:16).

A bead-based human inflammatory cytokine array was performed using LEGENDplex human inflammation panel 1 (BioLegend). Human primary macrophages were cultured for 24 h in the presence of S protein, with or without various doses of Roneparstat (50 to 200 μg/mL). Twenty-five microliters of the conditioned medium from various conditions was used to measure the secreted cytokines in each sample, according to the instructions from the manufacturer. The measurement was performed on a FACSCalibur flow cytometer (BD), and the data were analyzed using LEGENDplex software (BioLegend).

Gene expression analysis by quantitative PCR (qPCR) revealed a significant upregulation of HPSE, IL6, TNF, CCL2, and IFNG after the 51 protein challenge (FIG. 3B). To evaluate whether heparanase can regulate the expression of inflammatory cytokine genes such as IL-6, in parallel with the 51 protein challenge, we treated macrophages with various doses of active recombinant human heparanase for 12 h and examined the IL-6 expression level by qPCR. Treatment with exogenous heparanase induced elevated levels of IL-6 expression, at levels similar to those seen with 51 protein stimulation (FIG. 3C). Next, to examine whether HPSE blockade could suppress the induction of inflammatory cytokines, we challenged macrophages with 51 protein, in the presence or absence of various doses of Roneparstat. Indeed, treatment with Roneparstat resulted in a dose-dependent reduction in the expression of inflammatory cytokine genes such as IL6, TNF, CCL2, and IFNG (FIGS. 3D, 3E, 3F, and 3G). Using a flow cytometry-based multiplex inflammatory cytokine array, we showed that HPSE blockade with Roneparstat also significantly attenuated the protein levels of multiple inflammatory cytokines, including IL-6, TNF-α, IL-10, IL-1β, IL-23, IL-33, and IL-12p70 (FIGS. 3H, 3I, 3J, 3K, 3L, 3M. and 3N). In contrast, no significant changes were seen in interferon gamma (IFN-γ), IFN-α2, monocyte chemoattractant protein 1 (MCP-1), IL-8, IL-17A, and IL-18 (FIGS. 3O, 3P, 3Q, 3R, 3S, 3T, and 3U).

The results of these experiments demonstrated that 51 protein alone is sufficient to induce the upregulation of heparanase, which promotes the production of inflammatory cytokines. HPSE blockade with Roneparstat dampens inflammatory cytokine release induced by SARS-CoV-2 S1 protein in macrophages.

### Example 4: Sars-Cov-2 Spike Protein-Induced Inflammatory Cytokine Release is Partially Mediated by HPSE

To investigate the proinflammatory role of HPSE in macrophages in the context of SARS-CoV-2, the following experiments were conducted.

To knock down the HPSE gene in the human macrophage cell line THP-1, Lenti-X 293 cells were transfected with psPAX2, PMD2.G, and plasmid of interest using Lipofectamine 3000 (Invitrogen), and the supernatant was harvested at 24 h posttransfection. For HPSE KD, short hairpin RNA (shRNA) constructs in pLKO.1 puro vectors were obtained from Sigma-Aldrich (luciferase shRNA [shLuc], 5′-CAGAATCGTCGTATGCAGTGA-3′ (SEQ ID NO:17); HPSE shRNA 1, 5′-GAGGAGAAGTTACGGTTGGAA-3′ (SEQ ID NO:18); HPSE shRNA 2, 5′-CCCAAGAAGGAATCAACCTTT-3′ (SEQ ID NO:19); HPSE shRNA 3, 5′-GCGAGGAGATTCTGTAAACTT-3′) (SEQ ID NO:20). THP-1 cells were infected with lentivirus overnight in the presence of 10 μg/mL protamine sulfate. At 24 h postinfection, transduced cells were selected with 2 μg/mL puromycin for 3 days. RNA or conditioned medium was harvested from macrophages challenged with SARS-CoV-2 S1 protein (0.5 μg/mL) for 12 h or 24 h, respectively.

The HPSE gene in the human macrophage cell line THP-1 was knocked down using short hairpin RNA (shRNA) with a 60% average knockdown efficiency (FIG. 9A). THP-1 cell-derived macrophages were challenged with S1 protein (0.5 μg/mL). The knockdown of HPSE in THP-1 macrophages prevented the induction of IL6 or IL1B gene expression (FIGS. 5B and 5C). At the protein level, using a flow cytometry-based multiplex inflammatory cytokine array, we found marked increases in multiple inflammatory cytokines (IL-6, TNF-α, MCP-1, and IL-1B) in the conditioned medium from the 51-stimulated THP-1 cells but at much lower levels in the stimulated HPSE knockdown lines (FIGS. 5D, 5E, 5F, and 5G).

The results of these experiments that HPSE plays an important role in the induction of inflammation cytokines in macrophages upon 51 protein challenge.

### Example 5: Roneparstat Decreases Sars-Cov-2 Spike Protein-Induced Inflammatory Cytokine Release Via NF-KB Signaling

NF-KB signaling is a master regulator of inflammation, which regulates the transcription of several cytokines. To test whether the induction of the inflammatory cytokines by SARS-CoV-2 spike protein was dependent on NF-KB signaling, the following experiments were conducted.

Human macrophages were pretreated with the NF-KB inhibitor BAY-11-7082 (BAY) for 1 h, followed by the S1 protein challenge. Transcription levels of IL6, TNF, IL1B, and IFNG were assessed by qPCR using methods similar to those described above. Representative NF-KB (p65) immunostaining of human primary macrophages cotreated with S1 protein (0.5 μg/mL) and various doses of Roneparstat (50 and 100 μg/mL) for 24 h. Quantification of the mean fluorescence intensity (MFI) of the nuclear p65 signal. Seven to ten high-power fields were captured and quantified under each treatment condition. Western blots of cytoplasmic and nuclear p65 in human primary macrophages treated with various doses of Roneparstat (50 and 100 μg/mL) and S1 protein (0.5 μg/mL) were processed for 30 min. Blots were reprobed with an anti-TBP antibody as a loading control for nuclear fractions and β-actin for the cytoplasmic fractions. Cytoplasmic and nuclear p65 levels were quantified by NF-KB p65/β-actin and p65/TBP ratios, respectively. a.u., arbitrary units.

For Western blotting, nuclear and cytoplasmic protein lysates were isolated from human macrophages using the NE-PER nuclear and cytoplasmic extraction kit (Thermo Scientific) supplemented with a Halt protease inhibitor cocktail (Thermo Scientific) according to the manufacturer's protocol. Samples were separated on 4 to 20% Mini-Protean TGX precast protein gels (Bio-Rad) by SDS-PAGE and transferred onto Immobilon-P polyvinylidene difluoride (PVDF) membranes (EMD Millipore) overnight at 4° C. Membranes were incubated with NF-KB p65 (clone D14E12), TATA-binding protein (TBP) (clone D5C9H), or β-actin (clone 13E5) primary antibodies (1:1,000; Cell Signaling), followed by horseradish peroxidase (HRP)-conjugated anti-rabbit secondary antibody (1:2,000; Cell Signaling). All antibodies were diluted in 1× Tris-buffered saline (TBS)—Tween (TBST) with 5% bovine serum albumin (BSA). Bands were developed by enhanced chemiluminescence and quantified using ImageJ software.

For immunocytochemistry (ICC) staining, human PBMCs were resuspended in α-MEM (supplemented with 10% FBS and human recombinant M-CSF at 20 ng/mL) and seeded onto coverslips (number 1.5 coverslip, 13-mm glass diameter; VWR) in 24-well plates. Cells were cultured for 2 days, and the nonadherent cells were then removed by sequential washes with Dulbecco's PBS (DPBS) (Gibco). Fresh media with M-CSF were changed every 2 to 3 days until the cells reached 70 to 80% confluence (days 5 to 7). Cells were challenged with SARS-CoV-2 S1 (0.5 μg/mL) protein, with or without various doses of Roneparstat (50 μg/mL and 100 μg/mL), for 24 h. For immunostaining, each well was fixed with 4% paraformaldehyde (15 min at room temperature), followed by two washes with ice-cold TBS. Each coverslip was subsequently incubated with 0.2% Triton X-100 (Sigma) for 10 min, 10% goat serum (Cell Signaling) for 30 min, anti-human p65 primary antibody (1:700; Cell Signaling) overnight at 4° C., and Alexa Fluor 488-conjugated anti-rabbit antibody (1:400; Jackson ImmunoResearch) for 1 h, and nuclei were counterstained with 4′,6-diamidino-2-phenylindole (DAPI) (catalog number 422801; BioLegend) for 5 min. Coverslips were mounted onto glass slides (VWR) with ProLong Gold antifade mountant (catalog number P10144; Life Technologies).

For nuclear p65 quantification, slides were imaged with a confocal microscope (FluoView1200; Olympus) with a 40× water immersion lens objective. Seven to ten high-power fields were captured under each treatment condition. To quantify the nuclear p65 signal, captured images were analyzed using the Fiji imaging processing package with standard plug-ins. DAPI staining was used to determine nuclear segmentation and masking. Briefly, for each high-power field, binary image masks were created using a DAPI-positive single-channel image to define the nuclear region of interest (ROI). The image calculator was used to subtract the DAPI mask from the original p65 single-channel images to create images with p65 staining within the nuclear ROI. Image measurements of the p65 mean fluorescence intensities (MFIs) within the nuclear ROI were then determined by the mean gray value within the DAPI mask using an empirically derived threshold.

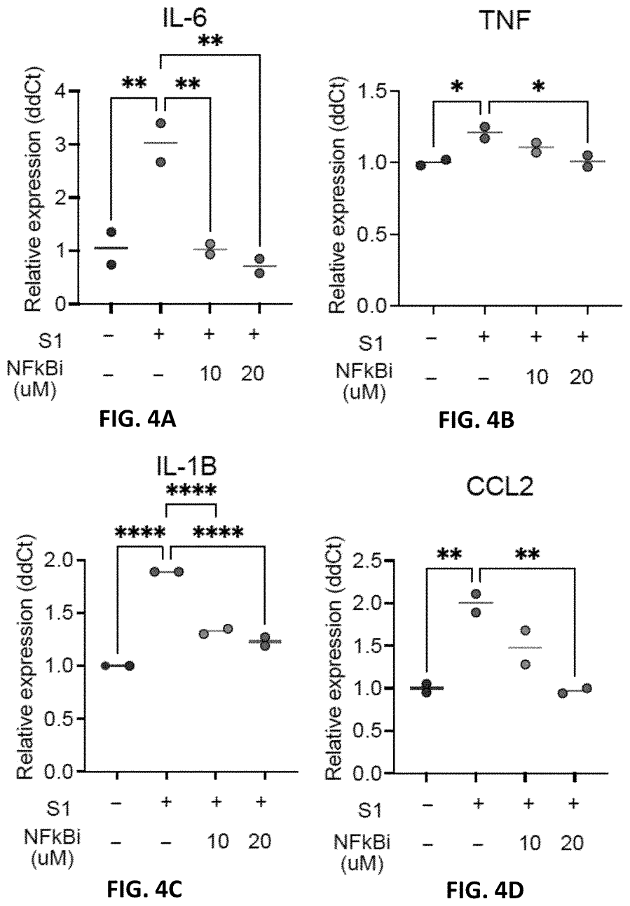

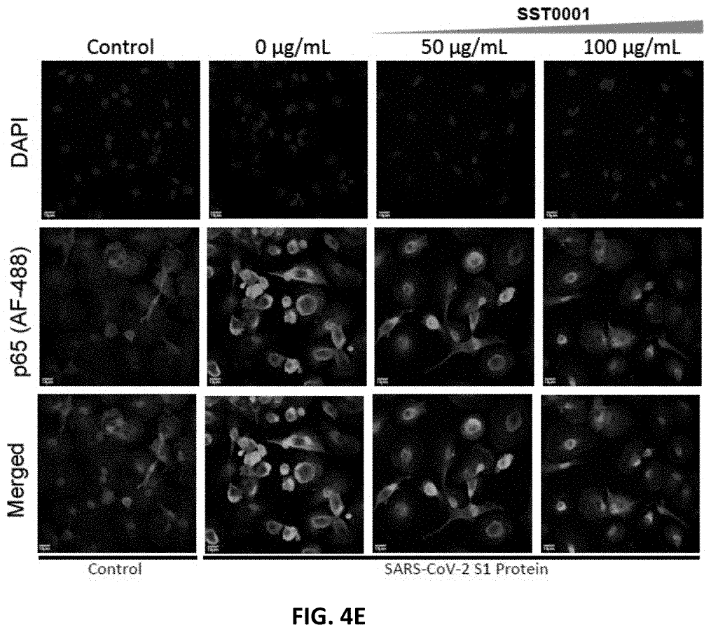

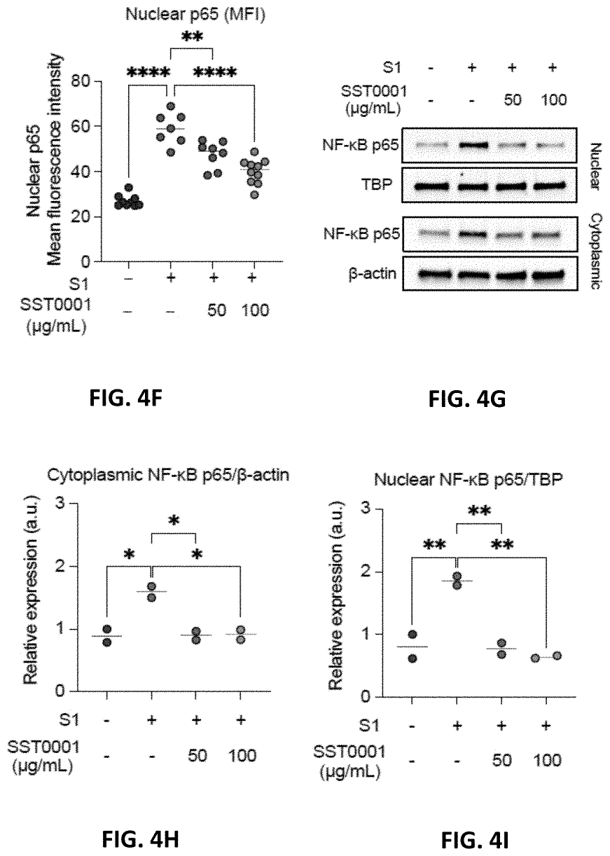

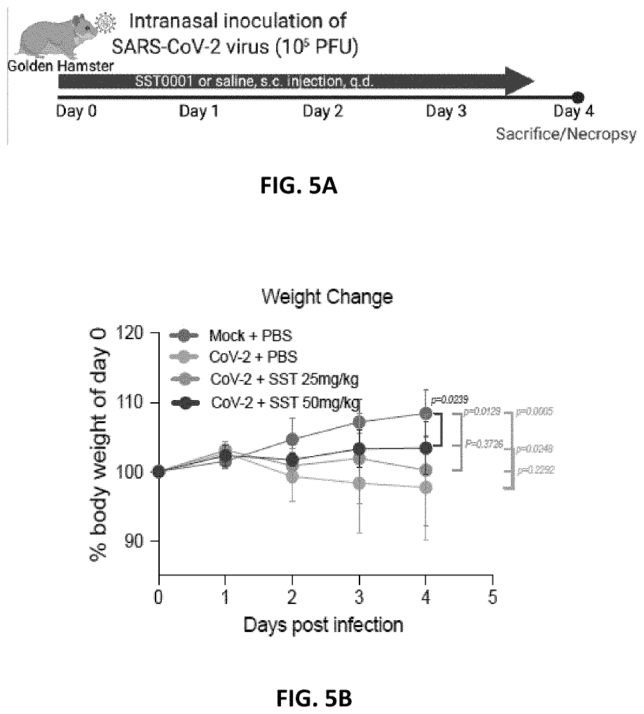

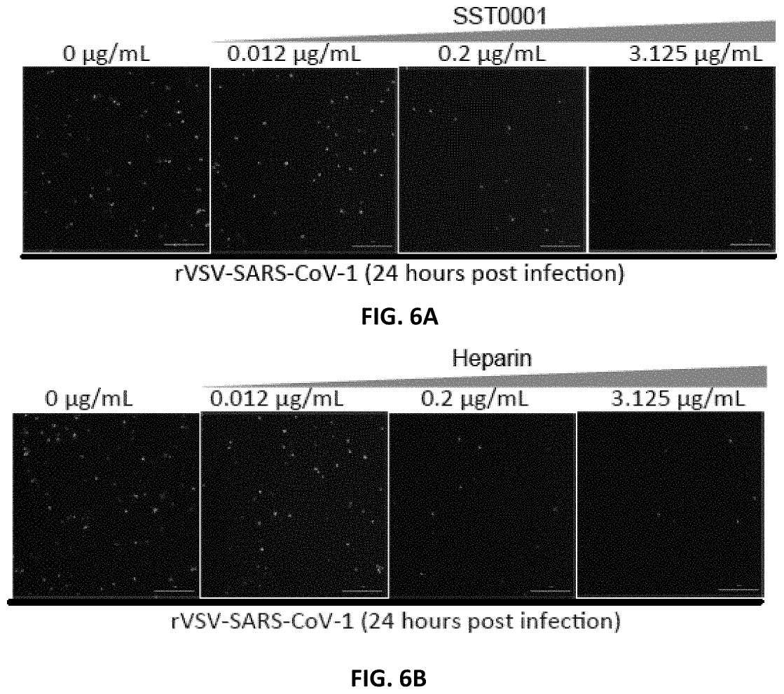

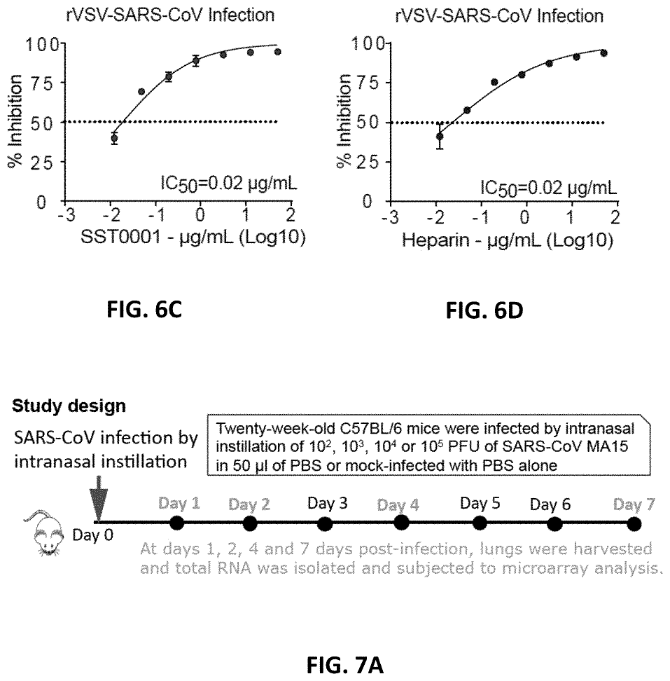

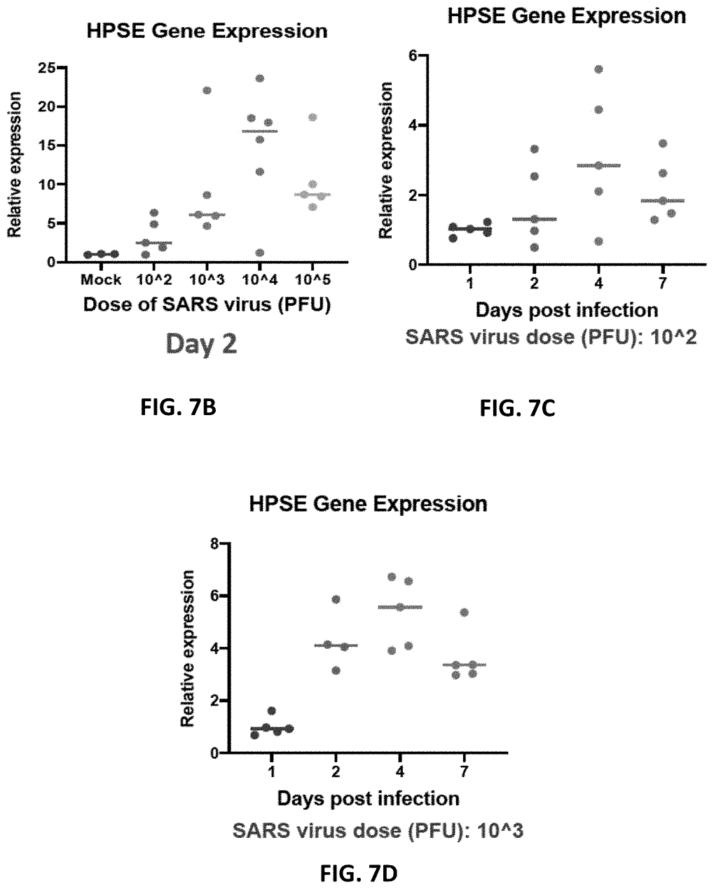

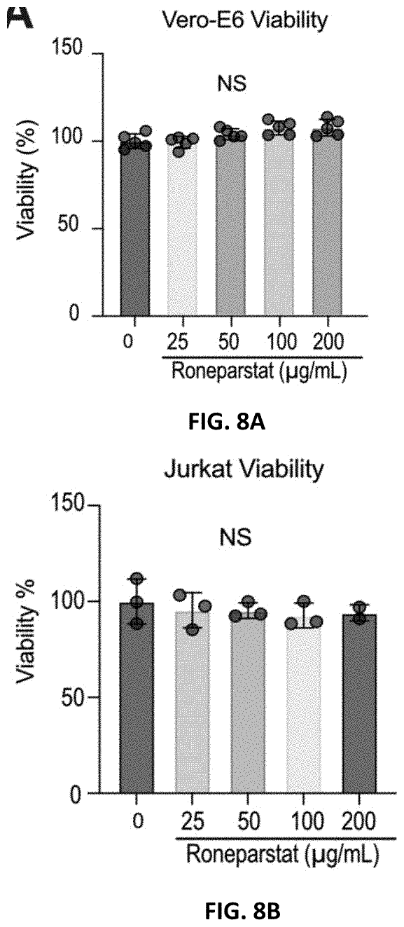

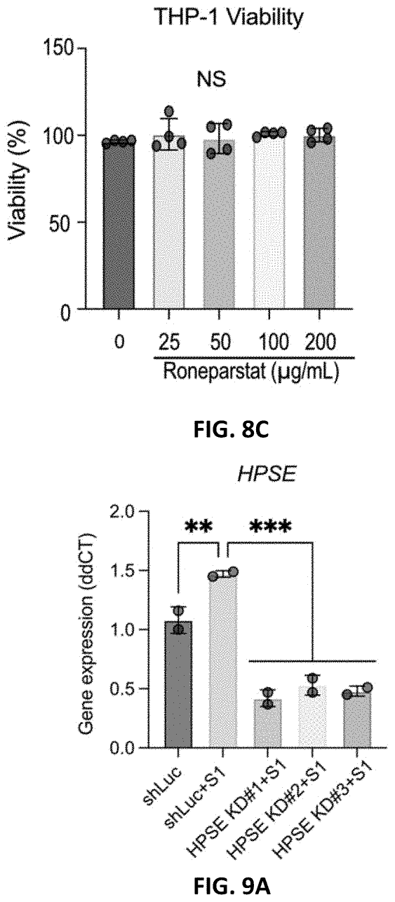

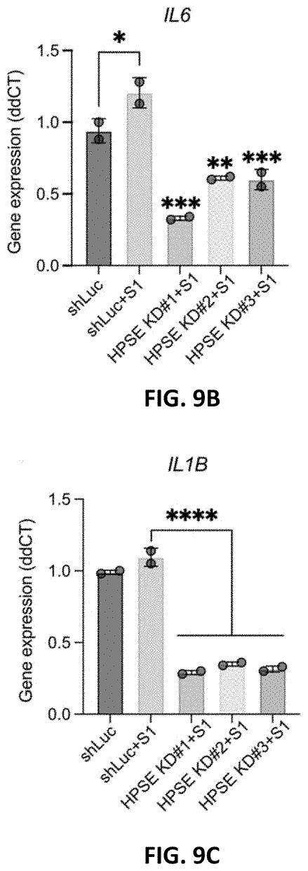

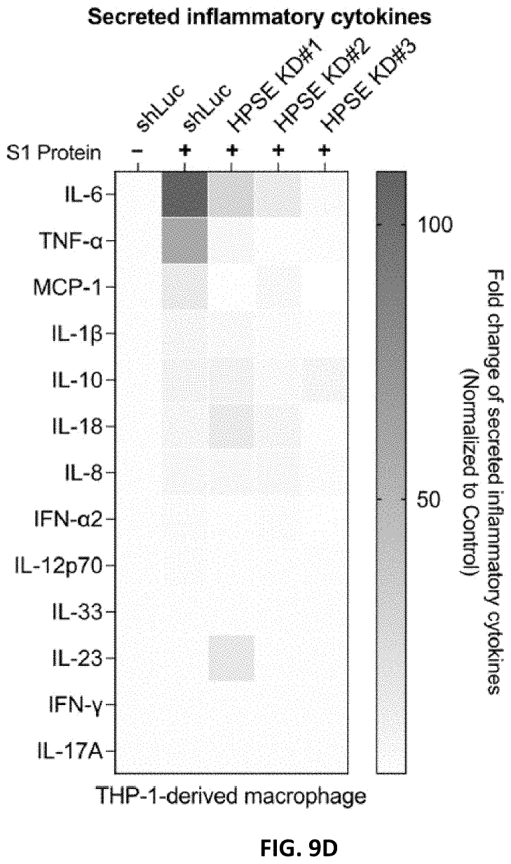

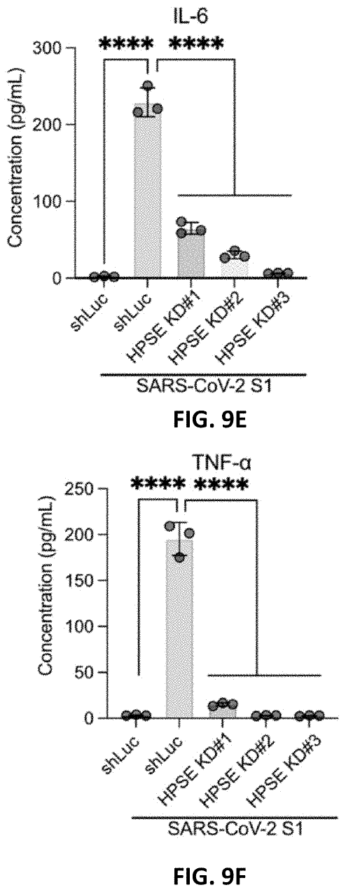

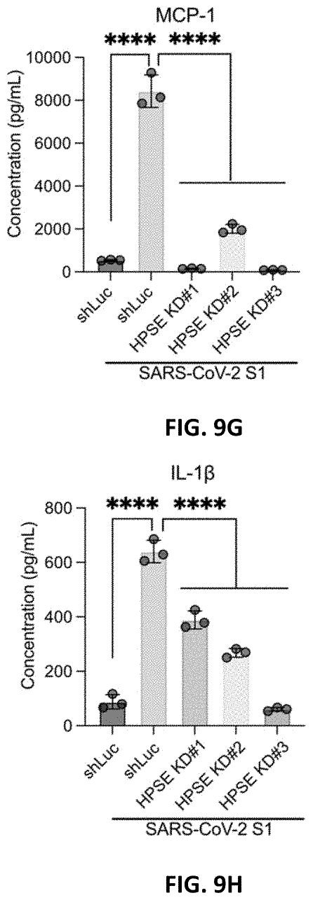

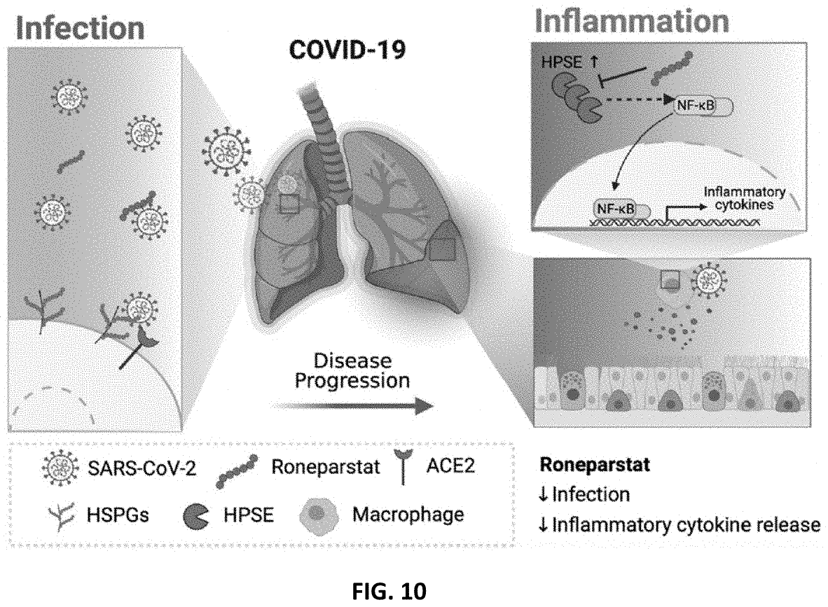

Treatment with BAY suppressed 51 protein-induced inflammatory cytokine gene expression (IL6, TNF, ILIB, and CCL2) in a dose-dependent manner (FIGS. 4A, 4B, 4C, and 4D). At a steady-state, cytosolic NF-KB p65 is retained in an inactive form and is targeted by E3 ligases for degradation. In response to proinflammatory signals, p65 translocates from the cytoplasm to the nucleus, where it is stabilized and activates the transcription of inflammatory cytokine genes. In macrophages, p65 also induces its own transcription, further increasing the nuclear occupancy of p65 and, consequently, cytokine production. Immunostaining of p65 in primary macrophages stimulated with 51 protein showed an overall increase of p65, with a significant increase in nuclear occupancy, as measured by the nuclear mean fluorescence intensity (MFI). Cotreatment with Roneparstat showed a dose-dependent reduction in nuclear p65 (FIGS. 4E and 4F). The reduction of nuclear and cytoplasmic NF-KB (p65) was also confirmed by Western blotting (FIGS. 4G, 4H, and 4I).

The results of these experiments demonstrated that in macrophages challenged with SARS-CoV-2 S1 protein, Roneparstat treatment reduced NF-KB-dependent induction of inflammatory cytokines.

### Example 6: Effect of Roneparstat Treatment on Histology and RNA Expression Profiles of Tissues in Golden Hamster Model

To assess the effect of a Roneparstat (SST0001) treatment of SARS-CoV-2 infection, the following experiments were conducted.

Twenty 4-week-old female Golden Syrian hamsters (Envigo, Indianapolis, Ind.) were randomly divided into 4 groups (n=5). Hamsters were initially housed in a BSL2 animal facility. Groups 1-3 were transferred into a BSL3 animal facility one day prior to the SARS-CoV-2-infection. Roneparstat (SST0001) was resuspended in PBS at 50 mg/mL and stored at room temperature.

As summarized in FIG. 5A, hamster groups 1, 2, and 3 were weighed and subcutaneously injected with 50 mg/kg, 25 mg/kg, and 0 mg/kg (PBS) of SST0001, respectively. Eight hours later the three groups were intranasally inoculated with 1.0×10⁵ PFU of SARS-CoV-2 in 40 μl DMEM. Group 4 housed in BSL2 animal facility was injected with PBS and inoculated with 40 μl DMEM as control. Each group was injected with indicated SST0001 or PBS at day 1, 2, and 3 post-infection, weighed and monitored daily. As illustrated in FIG. 5B, none of the hamster groups experienced a significant change in body mass over this observation period.

At day 4 post-infection, all hamsters were euthanized. The left lobe of lung, two pieces kidney tissues from both left and right kidneys, and nasal turbinate were collected and homogenized in PBS for both virus titration and/or RNA extraction. The right lobes of lung, the rest of kidneys, brain, liver, and spleen were fixed in 4% neutral buffered formaldehyde for histology and immunohistochemistry (IHC) analysis.

What is claimed is:

1. A method of treating an RNA virus infection in a subject in need, the method comprising administering a therapeutically effective amount of a heparanase inhibitor, wherein the RNA virus comprises SARS-CoV-1, SARS-CoV-2, HTLV-1, HIV-1, and any combination thereof.

2. The method of claim 1, wherein the heparanase inhibitor comprises heparin, a heparin mimetic, or any combination thereof.

3. The method of claim 2, wherein the heparanase inhibitor is Roneparstat.

4. The method of claim 1, wherein the administration of the therapeutically effective amount of the heparanase inhibitor results in inhibition of infection by the RNA virus and associated inflammatory cytokine production.

5. The method of claim 1, wherein the RNA virus is SARS-CoV-2.

6. The method of claim 1, wherein the RNA virus comprises an HSPG-dependent viral entry mechanism.


SEQUENCE LISTING (20 sequences)

SEQ ID NO: 1 - DNA, 22 bp, Artificial Sequence
  Synthetic sequence
ctctctgctctgccatctttag

SEQ ID NO: 2 - DNA, 21 bp, Artificial Sequence
  Synthetic Sequence
cctctggttgctatgaggttt

SEQ ID NO: 3 - DNA, 20 bp, Artificial Sequence
  Synthetic Sequence
cttccatccagttgccttct

SEQ ID NO: 4 - DNA, 23 bp, Artificial Sequence
  Synthetic Sequence
ctccgacttgtgaagtggtatag

SEQ ID NO: 5 - DNA, 21 bp, Artificial Sequence
  Synthetic Sequence
ccagggacctctctctaatca

SEQ ID NO: 6 - DNA, 20 bp, Artificial Sequence
  Synthetic Sequence
tcagcttgagggtttgctac

SEQ ID NO: 7 - DNA, 21 bp, Artificial Sequence
  Synthetic Sequence
tcatagcagccaccttcattc

SEQ ID NO: 8 - DNA, 24 bp, Artificial Sequence
  Synthetic Sequence
ctctgcactgagatcttcctattg

SEQ ID NO: 9 - DNA, 21 bp, Artificial Sequence
  Synthetic Sequence
atgtccaacgcaaagcaatac

SEQ ID NO: 10 - DNA, 20 bp, Artificial Sequence
  Synthetic Sequence
acctcgaaacagcatctgac

SEQ ID NO: 11 - DNA, 22 bp, Artificial Sequence
  Synthetic Sequence
agaatgtcttgattgtggaaga

SEQ ID NO: 12 - DNA, 21 bp, Artificial Sequence
  Synthetic Sequence
accttgaccatctttggatta

SEQ ID NO: 13 - DNA, 20 bp, Artificial Sequence
  Synthetic Sequence
caaaggcggccaggatataa

SEQ ID NO: 14 - DNA, 23 bp, Artificial Sequence
  Synthetic Sequence
ctagggattgagtccacattcag

SEQ ID NO: 15 - DNA, 21 bp, Artificial Sequence
  Synthetic Sequence
aggtcggtgtgaacggatttg

SEQ ID NO: 16 - DNA, 23 bp, Artificial Sequence
  Synthetic Sequence
tgtagaccatgtagttgaggtca

SEQ ID NO: 17 - DNA, 21 bp, Artificial Sequence
  Synthetic Sequence
cagaatcgtcgtatgcagtga

SEQ ID NO: 18 - DNA, 21 bp, Artificial Sequence
  Synthetic Sequence
gaggagaagttacggttggaa

SEQ ID NO: 19 - DNA, 21 bp, Artificial Sequence
  Synthetic Sequence
cccaagaaggaatcaaccttt

SEQ ID NO: 20 - DNA, 21 bp, Artificial Sequence
  Synthetic Sequence
gcgaggagattctgtaaactt